SEQUENCE LISTING (32 sequences)

SEQ ID NO: 1 - protein, 295 aa, Homo sapiens
  SIGNAL (1)..(24)
  mat_peptide (25)..(295): SARP-1
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 2 - protein, 271 aa, Homo sapiens
  mat_peptide (1)..(271): Q96HF1 without the signal peptide
LFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 3 - DNA, 882 bp, Homo sapiens
  CDS (265)..(882): SARP-1
gaattcgttcagcctggttaagtccaagctggctcattctgctcccccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttaaccagctccgtccctctaccccctaggggtcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactctcgntgcgtgcaggtgaaggatcgctgcgccccggtcatgtccgccttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgt

SEQ ID NO: 4 - protein, 206 aa, Homo sapiens
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSRCVQVKDRCAPVMSAFPWPDMLECDRFPQDNDLCIPLASSDHLLPATEEAPKVCEACKNKNDDDNDIMETLCKNDFALKIKVKEITYINR

SEQ ID NO: 5 - DNA, 1988 bp, Homo sapiens
  CDS (238)..(1125): SARP-1
ggctcattctgctcccccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttccccggctccgctccctctgccccctcggggtcgcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgagataccaaaatcatcctggagaccaagagcaagaccatttacaagctgaacggtgtgtccgaaagggacctgaagaaatcggtgctgtggctcaaagacagcttgcagtgcacctgtgaggagatgaacgacatcaacgcgccctatctggtcatgggacagaaacagggtggggagctggtgatcacctcggtgaagcggtggcagaaggggcagagagagttcaagcgcatctcccgcagcatccgcaagctgcagtgctagtcccggcatcctgatggctccgacaggcctgctccagagcacggctgaccatttctgctccgggatctcagctcccgttccccaagcacactcctagctgctccagtctcagcctgggcagcttccccctgccttttgcacgtttgcatccccagcatttcctgagttataaggccacaggagtggatagctgttttcacctaaaggaaaagcccacccgaatcttgtagaaatattcaaactaataaaatcatgaatatttttatgaagtttaaaaatagctcactttaaagctagttttgaataggtgcaactgtgacttgggtctggttggttgttgtttgttgttttgagtcagctgattttcacttcccactgaggttgtcataacatgcaaattgcttcaattttctctgtggcccaaacttgtgggtcacaaaccctgttgagataaagctggctgttatctcaacatcttcatcagctccagactgagactcagtgtctaagtcttacaacaattcatcattttataccttcaatgggaacttaaactgttacatgtatcacattccagctacaatacttccatttattagaagcacattaaccatttctatagcatgatttcttcaagtaaaaggcaaaagatataaattttataattgacttgagtactttaagccttgtttaaaacatttcttacttaacttttgcaaattaaacccattgtagcttacctgtaatatacatagtagtttacctttaaaagttgtaaaaatattgctttaaccaacactgtaaatatttcagataaacattatattcttgtatataaactttacatcctgttttacctataa

SEQ ID NO: 6 - protein, 295 aa, Homo sapiens; identical to SEQ ID NO: 1

SEQ ID NO: 7 - DNA, 1196 bp, Homo sapiens
  CDS (44)..(931): SARP-1
cccggctccgctccctctgccccctcggggtcgcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggtcaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgagataccaaaatcatcctggagaccaagagcaagaccatttacaagctgaacggtgtgtccgaaagggacctgaagaaatcggtgctgtggctcaaagacagcttgcagtgcacctgtgaggagatgaacgacatcaacgcgccctatctggtcatgggacagaaacagggtggggagctggtgatcacctcggtgaagcggtggcagaaggggcagagagagttcaagcgcatctcccgcagcatccgcaagctgcagtgctagtcccggcatcctgatggctccgacaggcctgctccagagcacggctgaccatttctgctccgggatctcagctcccgttccccaagcacactcctagctgctccagtctcagcctgggcagcttccccctgccttttgcacgtttgcatccccagcatttcctgagttataaggccacaggagtggatagctgttttcacctaaaggaaaagcccacccgaatcttgtagaaatattcaaactaataaaatcatgaatattttaa

SEQ ID NO: 8 - protein, 295 aa, Homo sapiens [sequence omitted]

SEQ ID NO: 9 - DNA, 1394 bp, Homo sapiens
  CDS (222)..(1109): SARP-1
ccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttccccggctccgctccctctgccccctcggggtcgcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggtcaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgagataccaaaatcatcctggagaccaagagcaagaccatttacaagctgaacggtgtgtccgaaagggacctgaagaaatcggtgctgtggctcaaagacagcttgcagtgcacctgtgaggagatgaacgacatcaacgcgccctatctggtcatgggacagaaacagggtggggagctggtgatcacctcggtgaagcggtggcagaaggggcagagagagttcaagcgcatctcccgcagcatccgcaagctgcagtgctagtcccggcatcctgatggctccgacaggcctgctccagagcacggctgaccatttctgctccgggatctcagctcccgttccccaagcacactcctagctgctccagtctcagcctgggcagcttccccctgccttttgcacgtttgcatccccagcatttcctgagttataaggccacaggagtggatagctgttttcacctaaaggaaaagcccacccgaatcttgtagaaatattcaaactaataaaatcatgaatatttttatgaagtttaaaaaaaaaaaa

SEQ ID NO: 10 - protein, 295 aa, Homo sapiens [sequence omitted]

SEQ ID NO: 11 - DNA, 1155 bp, Artificial
  Homo sapiens SARP-1(Fz)-Fc
atgctgcagggccctggctcgctgctgctgctcttcctcgcctcgcactgctgcctgggctcggcgcgcgggctcttcctctttggccagcccgacttctcctacaagcgcagcaattgcaagcccatcccggccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 385 aa, Artificial
  Synthetic Construct
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1062 bp, Artificial
  SARP-1(Fz)delta7-Fc
gacttctcctacaagcgcagcaattgcaagcccatcccggccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 354 aa, Artificial
  Synthetic Construct
DFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, Homo sapiens [sequence omitted]

SEQ ID NO: 16 - protein, 326 aa, Homo sapiens
  DOMAIN (1)..(98): IgG2 CH1 domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 290 aa, Homo sapiens
  DOMAIN (12)..(73): IgG3 hinge region
QMQGVNCTVSSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVQVHNAKTKPREQQFNSTFRVVSVLTVLHQNWLDGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 327 aa, Homo sapiens
  DOMAIN (1)..(98): IgG4 CH1 domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 31 aa, Homo sapiens
  SIGNAL (1)..(31): sFRP-1 signal peptide
MGIGRSEGGRRGAALGVLLALGAALLAVGSA

SEQ ID NO: 20 - protein, 24 aa, Homo sapiens
  SIGNAL (1)..(24): sFRP-2 (SARP-1) signal peptide
MLQGPGSLLLLFLASHCCLGSARG

SEQ ID NO: 21 - protein, 32 aa, Homo sapiens
  SIGNAL (1)..(32): sFRP-3 signal peptide
MVCGSPGGMLLLRAGLLALAALCLLRVPGARA

SEQ ID NO: 22 - protein, 18 aa, Homo sapiens
  SIGNAL (1)..(18): sFRP-4 signal peptide
MFLSILVALCLWLHLALG

SEQ ID NO: 23 - protein, 29 aa, Homo sapiens
  SIGNAL (1)..(28): sFRP-5 signal peptide
MRAAAAAGGVRTAALALLLGALHWAPARC

SEQ ID NO: 24 - DNA, 87 bp, Artificial
  mIgSP-tPA-pro signal peptide comprising the mouse immunoglobuline signal peptide (mIgSP) and the tissue plasminogen activator signal pro peptide (tPApro)
atgaagtgcagctgggtgatcttcttcctgatggccgtggtgaccggcgtgaattccagccaggagatccacgccaggttccgcaga

SEQ ID NO: 25 - DNA, 30 bp, Artificial
  hSARP-1 forward primer
gccaagcttcccacgatgctgcagggccct

SEQ ID NO: 26 - DNA, 30 bp, Artificial
  hSARP-1 reverse primer
gcgctcgagctagcactgcagcttgcggat

SEQ ID NO: 27 - DNA, 22 bp, Artificial
  Sarp1-B1p-121 forward primer
gcaggcttcgccaccatgctgc

SEQ ID NO: 28 - DNA, 34 bp, Artificial
  Sarp1-hFC-1036-R reverse primer
cacaagatttgggctcgcactgcagcttgcggat

SEQ ID NO: 29 - DNA, 37 bp, Artificial
  attB1-K forward primer
ggggacaagtttgtacaaaaaagcaggcttcgccacc

SEQ ID NO: 30 - DNA, 30 bp, Artificial
  attB2 reverse primer
ggggaccactttgtacaagaaagctgggtt

SEQ ID NO: 31 - DNA, 70 bp, Artificial
  5′-tPA-SARP primer
cggaattccagccaggagatccacgccaggttccgcagagacttctcctacaagcgcagcaattgcaagc

SEQ ID NO: 32 - DNA, 30 bp, Artificial
  3′FcSARPwt primer
ctagctagctttacccggagacagggagag

(19) United States
(12) Patent Application Publication
(10) Pub. No.: US 2010/0260785 A1
(43) Pub. Date: Oct. 14, 2010

(54) SARP-1 Fusion Proteins and Uses Thereof

(76) Inventors: Gabriela Saborio, Dingy En Vuache (FR); Maria Karmirantzou, Athens (GR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/744,492

(22) PCT Filed: Dec. 11, 2008
(86) PCT No.: PCT/EP08/67298

§ 371 Date: May 25, 2010

Related U.S. Application Data
(60) Provisional application No. 61/008,329, filed on Dec. 17, 2007.

(30) Foreign Application Priority Data
Dec. 13, 2007 (EP) 071231153

(51) Int. Cl. A61K 39/395; C07K 19/00; C07H 21/00; C12N 15/63; C12N 1/21; C12N 1/19; C12N 1/15; C12N 5/10; C12P 21/00; A61P 35/00; A61P 9/00; A61P 35/02

(52) U.S. Cl. 424/178.1; 530/391.1; 536/23.4; 435/320.1; 435/252.3; 435/254.2; 435/254.11; 435/325; 435/69.6

(57) ABSTRACT

The invention relates to a fusion protein comprising a mature SARP-1 polypeptide without the Netrindomain, the fusion protein further comprising an Fc region of an immunoglobulin, wherein the fusion protein lacks certain N-terminal amino acids of the mature SARP-1 polypeptide. The invention further relates to the use of said fusion protein for treating cancer, a fibrotic disorder or a cardiovascular disorder.

### FIELD OF THE INVENTION

The present invention is in the field of secreted apoptosis related proteins (SARPs) and uses thereof. More specifically, the invention relates to a fusion protein comprising a fragment of SARP-1 and an Fc region of an immunoglobulin, and the use of said fusion protein for the treatment and/or prevention of a fibrotic disorder, a cardiovascular disorder or cancer.

### BACKGROUND OF THE INVENTION

Secreted apoptosis related proteins (SARPs) constitute a family of secreted proteins of 280 to 346 amino acids and an estimated molecular weight of about 32 to 40 kDa, which are natural regulators of the Wnt signaling pathways. Structural characteristics of SARPs are a cysteine-rich domain (CRD) in the N-terminal half of the protein and a Netrin domain in the C-terminal half of the protein (Jones and Jomary, 2002).

The CRD of SARPs is 30-50% similar to the CRD of the protein family of Frizzled receptors. Therefore SARPs are also called secreted Frizzled-related proteins (sFRPs). The CRD of SARPs and Frizzled proteins is also called the Frizzled (FZ) domain. It comprises about 120 amino acids. The X-ray structure determination of a murine Frizzled protein CRD and a murine SARP CRD uncovered a new fold composed of mainly of alpha helices (Dann et al., 2001).

The Netrin (NTR) domain of SARPs shares sequence similarity with the axon guidance protein Netrin. This NTR module is defined by six cysteine residues and several conserved segments of hydrophobic residues (Banyai and Patthy, 1999). The function of the NTR domain in SARPs is unknown.

Initially three SARP proteins were identified in man: SARP-1 (SARP1, sFRP2, SDF-5, SFRP2), SARP-2 (SARP2, FRP, sFRP1, FrzA) and SARP-3 (SARP3, sFRP5, SFRP5) (WO 98/13493, WO 98/35043, Melkonyan et al., 1997). To date there are eight known members of this protein family, which additionally include sFRP3 (FrzB, Fritz, FRZB), sFRP4 (FrzB-2, SFRP4), Sizzled, Sizzled2 and Crescent. On the basis of the level of sequence identity sFRP1, sFRP2 and sFRP5 form a subgroup within the sFRP family, as do sFRP3 and sFRP4. Sizzled, Sizzled2 and Crescent form a third subgroup, which has not been identified in mammals (Kawano and Kypta, 2003).

Human SARP-1 (also known as sFRP-2 and SDF-5) is synthesized as a 295 amino acid precursor that contains a 24 amino acid signal peptide. Human SARP-1 is secreted as a 271 amino acid mature protein after cleavage of the signal peptide. According to the SwissProt protein database accession number Q96HF1 with the annotation of Nov. 13, 2007 the cysteine-rich (Frizzled) domain of SARP-1 comprises 122 amino acids (corresponding to amino acid 35 to 155 of SEQ ID NO:1), and the Netrin domain comprises 124 amino acids (corresponding to amino acid 172 to 295 of SEQ ID NO:1). Protein domain boundaries, such as those according to SwissProt protein database accession number Q96HF1, are generally based on predictions. In practice, the definition of the very minimal sequence of amino acids that make up a certain protein domain, such as the cysteine-rich (Frizzled) domain, may be refined over time based on further experimental data and/or protein domain prediction tools.

Mouse and rat SARP-1 have also been isolated and show 98% and 97% overall identity at the amino acid level to human SARP-1, respectively. Mouse and rat SARP-1 are 99% identical at the amino acid level. SARP-1 is believed to bind at least to Wnt1, 4, 7a, 8 and 9. Variants of human SARP-1 are known, which include without limitation the amino acid sequences shown in SEQ ID NOs: 1, 4, 6, 8 and 10.

The Frizzled (FZ) domain of SARPs mediates binding to the Wnt family of proteins (Rattner et al., 1997). By binding Wnts, SARP proteins can sequester Wnts away from the cell-surface receptors of Wnt and thereby, reduce the effective concentration of available Wnt protein. Thus, SARPs, are Wnt antagonists.

Wnts are 39-46 kDa cysteine-rich, secreted lipid-modified glycoproteins that are found in all multicellular organisms (metazoans) examined to date. Wnts act as short-range ligands to activate locally receptor-mediated signaling pathways. Wnts are expressed in spatially restricted and dynamic patterns in embryos and in adults.

Nineteen Wnts have been identified so far. They are grouped into two classes—canonical and non-canonical Wnts—on the basis of their activity in cell lines or in vivo assays. Canonical Wnts (e.g. Wnt1, Wnt3A and Wnt8) stabilize β-catenin, thereby activating transcription of T-cell factor (Tcf)/lymphocyte-enhancer-binding factor (LEF) target genes. Non-canonical Wnts (e.g. Wnt4, Wnt5A and Wnt11) activate other signaling pathways, such as the planar-cell-polarity (PCP)-like pathway that guides cell movements during gastrulation, and the Wnt/Ca²⁺ pathway.

Activation of the canonical β-catenin pathway leads to changes in gene expression that influence cell proliferation and survival, as well as cell fate. In addition to its roles during development, Wnt pathways play important roles in proliferation, differentiation and apoptosis in adult tissues.

Wnt signaling is initiated by the binding of a Wnt protein to the cysteine-rich domain of a cell surface receptor of the Frizzled family and a co-receptor of the low-density-lipoprotein-receptor-related-protein family (LRP5 or LRP6). The human and mouse genomes encode at least ten different Frizzled family members, which are presumed to have a partially promiscuous specificity for individual Wnt proteins.

Aberrant activation of the Wnt pathway has been found to occur during tumorigenesis. The frequent downregulation of SARP expression in carcinomas (Lee et al., 2004a), likely through increased methylation of the DNA coding for the SARP genes (Nojima et al., 2007), one the one hand and the upregulation of SARP expression in some degenerative diseases (Jones et al., 2000) on the other hand underpins their importance for homeostasis of healthy tissue by controlling Wnt activity. DNA methylation is an important means for controlling gene activity.

Hypermethylation generally leads to gene inactivation. Overexpression of sFRPs inhibited the proliferation of colorectal cancer and hepatocellular carcinoma cells (Shih et al., 2007; Suzuki et al., 2004).

SARP-1 (sFRP2) is highly expressed in canine mammary tumors but not in normal mammary glands (Lee et al., 2004b). DNA hypermethylation of the SARP-1 gene (sFRP2) has been observed in cells from up to 94% of stool probes from patients with colorectal cancer, as compared to 4% of control stool probes. SARP-1 is thus highly predictive for colorectal cancer (Huang et al., 2007a). (Huang et al., 2007b; Muller et al., 2004) DNA hypermethylation of the SARP-1 gene (sFRP2) has also been observed in gastric cancer (Cheng et al., 2007). These findings further support the notion that SARPs, and in particular SARP-1, are tumor suppressors that are inactivated by hypermethylation in tissue afflicted with colorectal, gastric and likely other cancers.

Inhibitors of Wnts, other than SARPs, have been shown to induce apoptosis in cancer cells. A monoclonal antibody against Wnt1 induced apoptosis in a variety of human cancer cell lines, including non small cell lung cancer, breast cancer, mesothelioma and sarcoma cells (Batra et al., 2006; He et al., 2004), and also in colorectal cancer cell lines (He et al., 2005) and fresh primary cultures of lung metastasis of sarcoma (Mikami et al., 2005). The soluble naturally occurring Wnt inhibitory factor-1 (WIF-1) induced apoptosis in colorectal cancer cell lines (He et al., 2005), and WIF-1 expression in vivo resulted in tumor growth suppression by inhibition of Wnt signaling (Lin et al., 2007).

WO 2006/055635 relates to methods for inhibiting growth of a tumor cell with a compound that alters Wnt signaling, such as a Wnt antagonist or an antagonist of the Wnt receptor. The only antagonist disclosed in WO 2006/055635 is a siRNA specific for low density lipoprotein receptor-related protein 5 or 6.

WO 2005/033048 discloses that Wnt signaling pathways can be inhibited with certain aromatic small molecule compounds. The compounds disclosed in WO 2005/033048 do not bind to Wnt or inhibit Wnt binding to the Frizzled receptor.

EP1733739 relates to agents that enhance the expression and/or activity of SFRPs through the modulation of the Discs large (Dig) gene, and the use of said agents for preventing or treating cancer. The Dig gene was originally identified as a tumor suppressor gene in Drosphila.

SARPs also have a role in the homeostasis of other tissue. SARP-1 mediates myocardial survival and repair by increasing cellular β-catenin expression in hypoxic cardiomyocytes (Mirotsou et al., 2007). β-catenin expression protects cardiomyocytes against ischemic injury.

The canonical Wnt (β-catenin pathway) is aberrantly activated in fibrosis (Chilosi et al., 2003), and SARP-1 has been shown to protect mice from bleomycin-induced pulmonary fibrosis and thus to be useful for the treatment of scleroderma and other fibrotic diseases (WO 02/46225). WO 02/46225 also discloses that fragments of SARP-1 comprising the Frizzled domain are useful for the treatment of scleroderma and other fibrotic diseases. In a disease model of renal fibrosis the progression of the disease could be suppressed with the administration of sFRP4, which is a member of the SARP protein family (Surendran et al., 2005).

There is therefore a need for antagonists of Wnt signaling that are useful in therapeutic applications. SARPs are effective Wnt antagonists in vivo. They inhibit canonical and non-canonical Wnt pathways. As they are naturally occurring proteins, SARPs and derivatives thereof have a low risk of being toxic in humans or mammals.

There is therefore a need for SARPs, such as SARP-1, or variants, or derivatives, or biologically active fragments of SARPs or SARP-1, which have improved characteristics.

Improved characteristics may not only relate directly to the biological activity of the variant or derivative of SARPs or SARP-1, but also to other aspects, which are relevant for therapeutic proteins, such as e.g. in vivo half-life or pharmacokinetics, routes of delivery or characteristics with regard to the manufacturing, such as the protein expression efficiency in cell culture systems. The immunogenicity is another critical aspect of therapeutic proteins, such as SARP-1, variants, derivatives, or biologically active fragments thereof. Therapeutic proteins, in particular if they are non-endogenous proteins, i.e. proteins that do not have a counterpart with identical amino acid sequence in the human or animal body to be treated, but even if they have such counterpart, frequently elicit an immune response against the therapeutic protein when administered to the human or animal body, which immune response may lead to unwanted side effects and/or reduced biological activity or therapeutic efficiency of the therapeutic protein. An improved characteristic of a SARP-1 variant, derivative, or biologically active fragment thereof may therefore reside in an essentially unaltered or reduced immunogenicity of the SARP-1 variant, derivative, or biologically active fragment thereof as compared to the endogenous SARP-1 polypeptide.

Fc fusion proteins are well known in the art. Fc fusion proteins are chimeric polypeptides consisting of the Fc region of an immunoglobulin heavy chain fused to an unrelated protein or protein fragment. For application in humans typically the Fc region of a human immunoglobulin heavy chain is used. When the single fusion polypeptide chain comprising the Fc region and the unrelated protein or protein fragment is expressed in a cell, it generally forms a dimer with a second Fc region containing polypeptide through the formation of disulfide bonds, analogous to the formation of heavy chain dimers in an immunoglobulin. It is, however, possible to construct Fc fusion proteins that comprise only one copy of the polypeptide chain of the unrelated protein (Dumont et al., 2006).

A native immunoglobulin molecule consists of two identical heavy chains, and two identical light chains. The heavy chain constant region includes CH₁, the hinge region, CH₂, and CH₃. Papain digestion of antibodies produces two fragments, Fab and Fc. The Fc fragment consists of CH₂, CH₃, and part of the hinge region. In human IgG molecules, the Fc fragment is generated by papain cleavage of the hinge region N-terminal to Cys-226. Therefore, the human IgG heavy chain Fc region was initially defined as stretching from the amino acid residue at position 226 to the C-terminus. The above numbering is according to Kabat (Kabat, 1988), who had based it on the sequence of a myeloma protein later identified as the immunoglobulin EU (Edelman et al., 1969). The term “Fc region” or “Fc fragment” as used hereinafter and further explained below may comprise not only the partial but also the complete hinge region or no hinge region at all.

It has been recognized that the Fc region is critical for maintaining the serum half-life of an immunoglobulin of class G (IgG) (Ward and Ghetie, 1995). Studies have found that the serum half-life of an IgG is mediated by binding of Fc to the neonatal Fc receptor (FcRn). FcRn is a heterodimer consisting of a transmembrane a chain and a soluble β-chain (β₂-microglobulin). The α₁ and α₂ domains of FcRn interact with the CH₂ and CH₃ domains of the Fc region. The site on the Fc fragment of human IgG that interacts with FcRn has been mapped (Kim et al., 1999; Vaughn et al., 1997).

The correlation between the affinity for FcRn binding and the serum half-life of an immunoglobulin is well known in the art (Datta-Mannan et al., 2007b). Significantly, such a correlation has been extended to engineered antibodies with higher affinity for FcRn than their wild-type parent molecules. A large number of publications and patents based upon mutagenesis studies support this correlation (Ward and Ghetie, 1995; Ghetie et al., 1997; Dall'Acqua et al., 2002; Hinton et al., 2004; Hinton et al., 2006; Shields et al., 2001; Datta-Mannan et al., 2007a; Kamei et al., 2005, U.S. Pat. No. 6,165,745, U.S. Pat. No. 6,277,375, US 2002/009819, WO 97/34621, WO 98/05787, WO 02/060919, WO 04/035752 and WO 2005/037867).

The Fc region can also be used to achieve oral or pulmonary delivery of therapeutic proteins. Fc fusion proteins have been successfully delivered via these routes (Bitonti and Dumont, 2006; Bitonti et al., 2004; Low et al., 2005; Dumont et al., 2005).

Methods for fusing or conjugating polypeptides to the constant (Fc) regions of antibodies (i.e. for making Fc fusion proteins) are well known in the art and are described in, for example without limitation in WO 2005/037867.

### SUMMARY OF THE INVENTION

The invention provides a fusion protein comprising a SARP-1 polypeptide without the Netrin domain, the fusion protein further comprising the Fc region of an immunoglobulin heavy chain, wherein the fusion protein is characterized in that it additionally lacks the N-terminal amino acids nos. 1-10, 1-9, 1-8, 1-7, 1-6, 1-5 or 1-4 of a mature SARP-1 polypeptide. The above fusion protein is referred to hereinafter as SARP-1(Fz)deltaN-Fc.

The invention is based on the finding that a SARP-1 polypeptide or a SARP-1 fusion protein, e.g. a SARP-1 fusion protein comprising a mature SARP-1 polypeptide without the Netrin domain and comprising the Fc region of an immunoglobulin heavy chain, is expressed only at low levels in protein expression systems known in the art, such as HEK or CHO cells.

Furthermore, it was found by the present inventors that said SARP-1 polypeptides or SARP-1 fusion proteins are not expressed in such expression systems with the N-terminus as encoded by the host cell vector but with N-terminal deletions of varying degrees, such that the polypeptides or fusion proteins expressed were not uniform. This phenomenon was referred to as ragging. It is highly desirable, however, inter alia for regulatory purposes that compositions of proteins that are to be used for therapeutic applications are uniform and exhibit batch-to-batch consistency. There is thus a need for a biologically active SARP-1 variant or derivative that is amenable to efficient manufacturing processes with improved batch-to-batch consistency.

It has also been determined by the present inventors that during the manufacture of (i) SARP-1 polypeptide, or (ii) a fusion protein comprising full length mature SARP-1 and the Fc region of an immunoglobulin heavy chain (SARP-1-Fc), or (iii) a fusion protein comprising a mature SARP-1 polypeptide without the Netrin domain and comprising the Fc region of an immunoglobulin heavy chain (SARP-1(Fz)-Fc), in all three instances the polypeptide product expressed in cell culture formed to a high degree high molecular weight complexes, which could not be dissolved. Due to loss of the desired polypeptide in insoluble complexes the overall yield of active polypeptide that could be used for therapeutic purposes was low.

Surprisingly it was now found by the inventors that the expression level in cell culture was significantly higher for a fusion protein comprising a mature SARP-1 polypeptide without the Netrin domain, the fusion protein further comprising the Fc region of an immunoglobulin heavy chain, wherein the fusion protein is characterized in that the N-terminus as encoded by the host cell vector lacked a defined number of amino acids of a mature SARP-1 polypeptide, preferably the N-terminal amino acids 1-10, 1-9, 1-8, 1-7, 1-6, 1-5 or 1-4, as compared to the same fusion protein not lacking said N-terminal amino acids. The polypeptide expression level of said polypeptide lacking N-terminal amino acids was about 2× higher when transiently expressed in HEK cells than the expression level of the polypeptide with the N-terminal amino acids of mature SARP-1 under the same cell culture conditions.

Even more surprisingly said fusion protein lacking a defined number of N-terminal amino acids did not exhibit any ragging; i.e. was produced with the N-terminus as encoded by the host cell vector. More than 90%, and in many instances 100% of said polypeptide produced, did not show any N-terminal deletion. In contrast when the corresponding fusion protein with the N-terminal amino acids of mature SARP-1 was produced under the same cell culture conditions, the polypeptide produced exhibited an N-terminus with varying deletions.

Furthermore, it was found by the present inventors that at least the N-terminal amino acids nos. 1-10 of the mature SARP-1 polypeptide; i.e. at least the amino acids LFLFGQPDFS of mature human SARP-1, could be deleted without reducing the biological activity of the SARP-1 fusion protein as compared to the fusion protein without the N-terminal deletion.

The Fc region comprised in the SARP-1 fusion protein of the invention can for example without limitation confer an increased in vivo half-life, better pharmacokinetics or the possibility to administer the fusion protein via other routes such as oral or pulmonary routes as it is known in the art.

Furthermore, the fusion protein comprising a mature SARP-1 polypeptide without the Netrin domain, and the fusion protein further comprising the Fc region of an immunoglobulin heavy chain, wherein the fusion protein is characterized in that the N-terminus as encoded by the host cell vector lacked a defined number of amino acids of a mature SARP-1 polypeptide preferably the N-terminal amino acids 1-10, 1-9, 1-8, 1-7, 1-6, 1-5 or 1-4, does not exhibit an increased immunogenicity as compared to the same fusion protein that does not lack said N-terminal amino acids.

The instant invention thus provides a SARP-1 fusion protein lacking N-terminal amino acids of mature SARP-1, preferably the N-terminal amino acids 1-10, 1-9, 1-8, 1-7, 1-6, 1-5 or 1-4, that can be manufactured with higher yield, in higher purity and with better batch-to-batch consistency while maintaining the biological activity of SARP-1 as compared to SARP-1 proteins known in the art.

Accordingly, one embodiment of the invention is a fusion protein comprising a mature SARP-1 polypeptide without the Netrin domain, the fusion protein further comprising the Fc region of an immunoglobulin heavy chain, wherein the fusion protein is characterized in that it lacks the N-terminal amino acids nos. 1-10, 1-9, 1-8, 1-7, 1-6, 1-5 or 1-4 of the mature SARP-1 polypeptide.

Another embodiment of the invention is polynucleotide encoding a fusion protein comprising a mature SARP-1 polypeptide without the Netrin domain, the fusion protein further comprising the Fc region of an immunoglobulin heavy chain, wherein the fusion protein is characterized in that it lacks the N-terminal amino acids nos. 1-10, 1-9, 1-8, 1-7, 1-6, 1-5 or 1-4 of the mature SARP-1 polypeptide.

Another embodiment of the invention is a fusion protein according to the first, second or third embodiment for the treatment of cancer, a fibrotic disorder or a cardiovascular disorder.

Even another embodiment of the invention is a pharmaceutical composition comprising a fusion protein according to the first, second or third embodiment as active ingredient, optionally together with a pharmaceutically acceptable carrier or excipient.

### BRIEF DESCRIPTION OF THE FIGURES

FIG. 1: The figure shows an alignment of SARP-1 variants according to SwissProt accession no. Q96HF1 and GenBank accession nos. AF311912; AY359001, BC008666 and AF017986 generated with the software Multalin version 5.4.1 (Corpet, 1988). Variable amino acid positions are underlined and marked in bold. The amino acids corresponding to the signal peptide, the Frizzled domain and the Netrin domain are indicated in boxes. The domain boundaries are taken from SwissProt protein database accession number Q96HF1 with the annotation of Nov. 13, 2007.

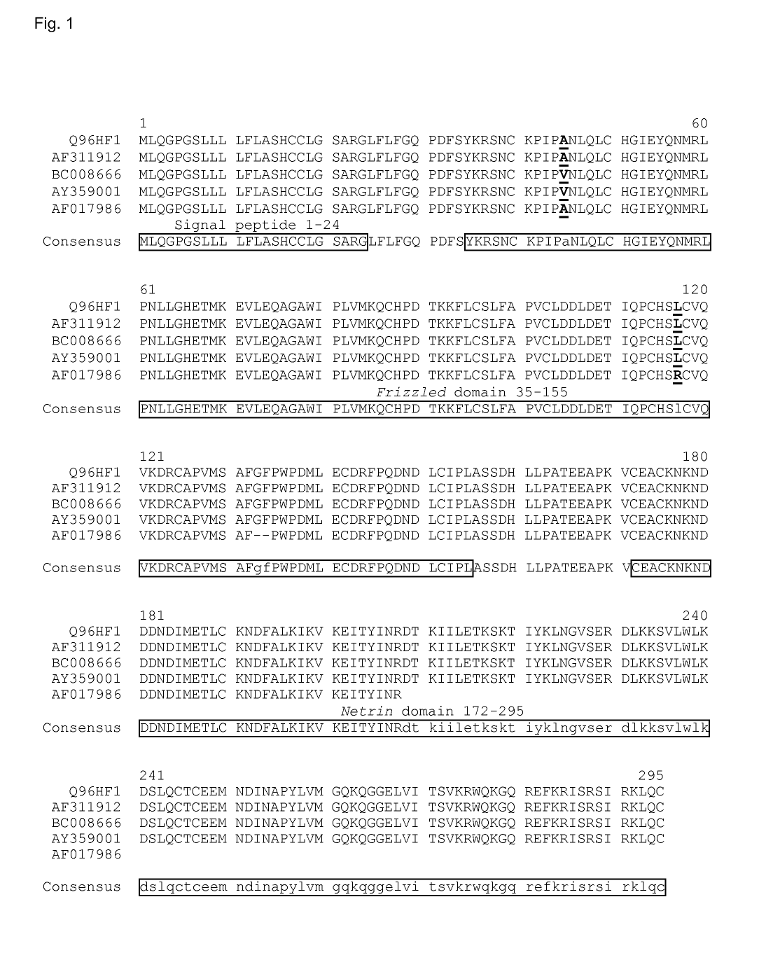

FIG. 2: SARP-1(Fz)deltaN-Fc is a fusion protein comprising two parts, the N-terminal part comprising a mature SARP-1 polypeptide without the Netrin domain and the C-terminal part comprising that constant domain (consisting of the hinge region, CH₂ and CH₃ domains) of an immunoglobulin heavy chain. SARP-1(Fz)deltaN-Fc lacks N(N=4, 5, 6, 7, 8, 9 or 10) amino acids of the mature SARP-1 polypeptide of SEQ ID NO:2 at the N-terminus.

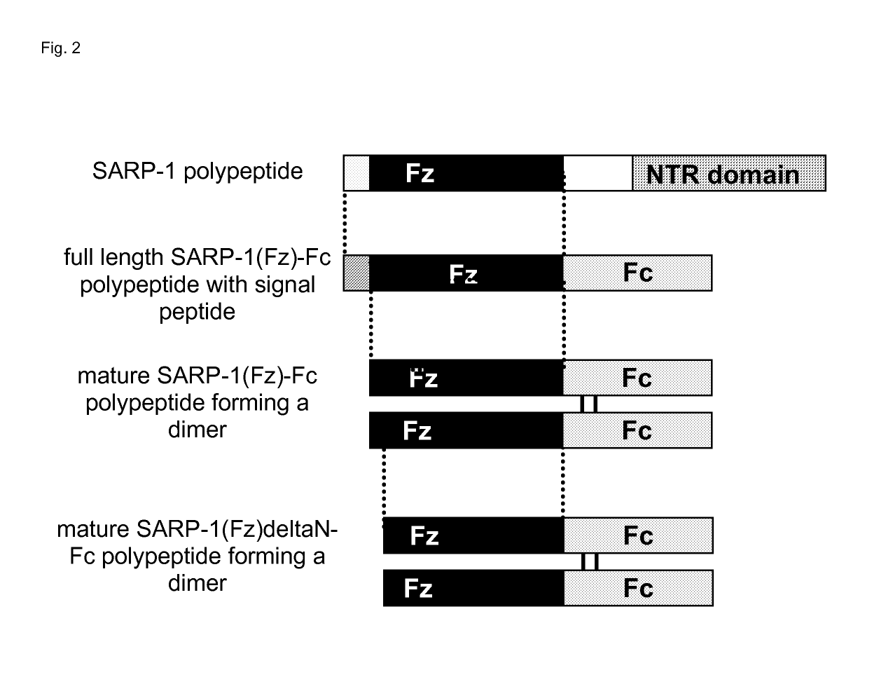

FIG. 3: SARP-1(Fz)delta7-Fc inhibited Wnt 1 and Wnt 2 activation in a reporter cellular assay. The Eₘₐₓ reached is 80%. The EC₅₀ is 0.75 μM. SARP-1(Fz)delta7-Fc lacks 7 amino acids at the N-terminus.

FIG. 4: SARP-1(Fz)delta7-Fc, applied at a concentration of 10.0 μg/ml for 2, 4 or 6, significantly decreased PDGF-induced cell growth of hepatic stellate cells. The experiment is described in Example 5.

FIG. 5: SARP-1(Fz)delta7-Fc inhibited cell proliferation of the mouse melanoma cell line B16F1 as described in Example 6.

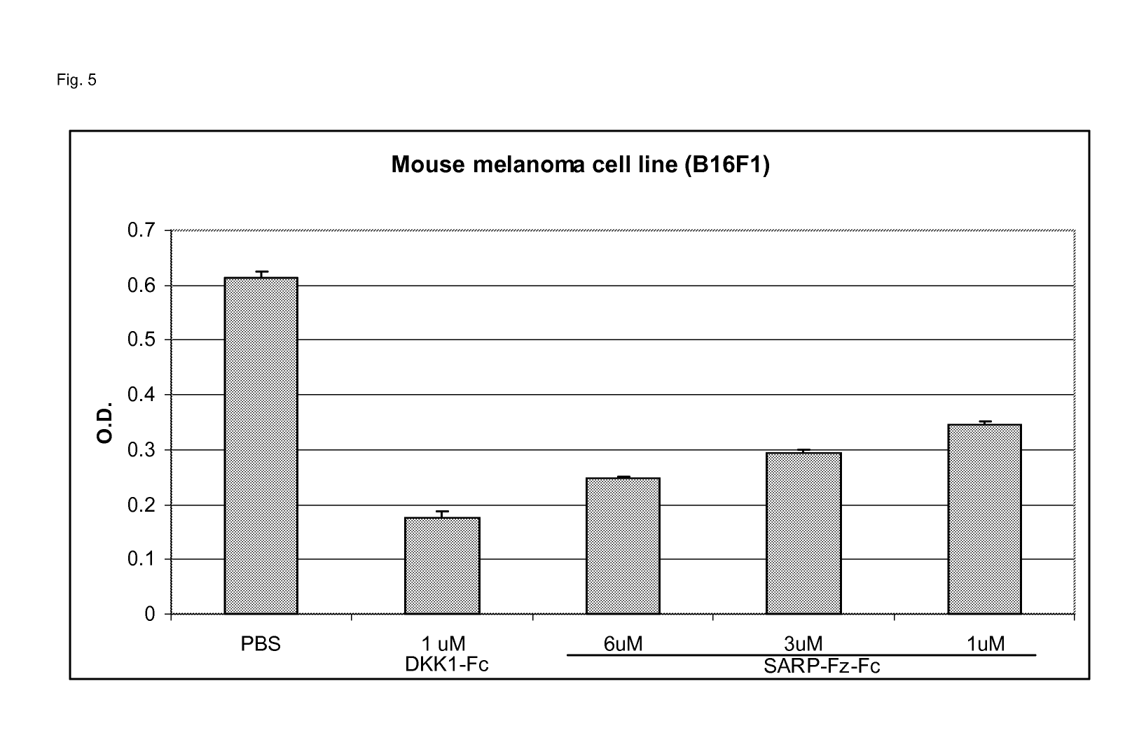

FIG. 6: SARP-1(Fz)delta7-Fc inhibited cell proliferation of the human colon carcinoma cell line SW480 as described in Example 6.

FIG. 7: SARP-1(Fz)delta7-Fc inhibited cell proliferation of the human colon carcinoma cell line SW620 as described in Example 6.

FIG. 8: SARP-1(Fz)delta7-Fc decreased alfa smooth muscle actin (α-SMA) accumulation in the unilateral ureter obstruction (UUO) murine model of renal fibrosis as described in Example 7. Alfa smooth muscle actin is a marker of the pathological transformation of the normal fibroblasts to myofibroblast, considered as a key player in the collagen overproduction in renal fibrosis, among other fibrotic conditions. The reduction of this marker by SARP-1(Fz)delta7-Fc indicates a favourable effect avoiding this pathological transformation and therefore decreasing collagen production.

FIG. 9: SARP-1(Fz)delta7-Fc reduced BrdU incorporation of fibroblast from patients with interstitial pulmonary fibrosis (IPF) in the basal and PDGF condition as described in Example 8.

### DETAILED DESCRIPTION OF THE INVENTION

The invention thus provides a fusion protein comprising a mature SARP-1 polypeptide without the Netrin domain, the fusion protein further comprising the Fc region of an immunoglobulin heavy chain, wherein the fusion protein is characterized in that it additionally lacks the N-terminal amino acids nos. 1-10, 1-9, 1-8, 1-7, 1-6, 1-5 or 1-4 of the mature SARP-1 polypeptide. The above fusion protein is referred to hereinafter as SARP-1(Fz)deltaN-Fc.

The first embodiment of the invention is a fusion protein comprising a mature SARP-1 polypeptide without the Netrin domain, the fusion protein further comprising the Fc region of an immunoglobulin heavy chain, wherein the fusion protein is characterized in that it lacks the N-terminal amino acids nos. 1-10, 1-9, 1-8, 1-7, 1-6, 1-5 or 1-4 of the mature SARP-1 polypeptide.

The second embodiment of the invention is a fusion protein comprising the mature SARP-1 polypeptide of SEQ ID NO:1, 4, 6, 8 or 10 without the Netrin domain, the fusion protein further comprising the Fc region of an immunoglobulin heavy chain, wherein the fusion protein is characterized in that it lacks the N-terminal amino acids nos. 1-10, 1-9, 1-8, 1-7, 1-6, 1-5 or 1-4 of the mature SARP-1 polypeptide.

The third embodiment of the invention is a fusion protein according to the second embodiment of the invention, wherein the mature SARP-1 polypeptide without the Netrin domain derives from a SARP-1 variant, which variant has at least 99%, 98%, 97%, 96%, 95%, 94%, 93%, 92%, 91%, 90%, 85%, 80%, 75% or 70% identity with the mature SARP-1 polypeptide of SEQ ID NO:1, 2, 4, 6, 8 or 10, and which variant has at least one of the biological activities of SARP-1.

The fourth embodiment of the invention is a fusion protein comprising amino acids nos. 35 to 153 of SEQ ID NO:6, 8 or 10, or comprising amino acids nos. 35 to 151 of SEQ ID NO:4, and the fusion protein further comprising the Fc region of an immunoglobulin heavy chain.

The Fc region according to the above embodiments may be derived from the heavy chain of any immunoglobulin class (i.e. IgG, IgM, IgA, IgE or IgD), but preferentially from IgG, such as IgG₁, IgG₂, IgG₃, or IgG₄. Exemplary amino acid sequences for the Fc regions of IgG₁, IgG₂, IgG₃, or IgG₄ are shown in SEQ ID NOs:15, 16, 17 and 18, respectively. It should be noted that there are allelic variants of IgG₁, IgG₂, IgG₃, or IgG₄ known in the art. The Fc region of any of them may be comprised in the fusion protein according to the above embodiments. The most preferred Fc region is the IgG₁ Fc region. An exemplary amino acid sequences for the IgG₁ Fc region is shown in SEQ ID NO:14 from amino acid 123 to 354. A further suitable Fc region is an IgG Fc region which is latest 99%, 98%, 97%, 96%, 95%, 94%, 93%, 92%, 91%, 90%, 85% or 80% identical to one the Fc regions of SEQ ID NOs:15, 156, 17 and 18 or in SEQ ID NO:14 from amino acid 123 to 354.

In a further embodiment the IgG₁, IgG₂, IgG₃ or IgG4 heavy chain Fc region comprises at least one mutation of an amino acid in order to reduce or increase any potential complement activation or antibody-dependent cellular cytotoxicity (ADCC) elicited by the Fc fusion protein, or in order to modulate (i.e. increase or decrease) the binding affinity of the Fc fusion protein to the neonatal Fc receptor (FcRn). Increased affinity of the Fc fusion protein to the FcRn will lead to increased in vivo half-life of the Fc fusion protein, and also to increased uptake of the Fc fusion protein via oral or pulmonary routes.

The mutations in the Fc region that are required to reduce complement activation or ADCC elicited by the Fc fusion protein, in order to modulate (i.e. to increase or decrease) the in vivo half-life of the Fc fusion protein, or in order to make the Fc fusion protein amenable to oral or pulmonary delivery are known in the art.

A further embodiment of the invention is a variant of SARP-1(Fz)deltaN-Fc which has at least 99%, 98%, 97%, 96%, 95%, 94%, 93%, 92%, 91%, 90%, 85%, 80%, 75% or 70% identity with the polypeptide of SEQ ID NO:14, lacks the N-terminal amino acids nos. 1-10, 1-9, 1-8, 1-7, 1-6, 1-5 or 1-4 of the mature SARP-1 polypeptide, and further has at least one of the biological activities of SARP-1.

A further embodiment of the invention is a mutein of SARP-1(Fz)deltaN-Fc in which not more than 1, 2, 3, 4, 5, 6, 7, 8, 9, 10, 15, 20, 25, 30, 35, 40, 50, 100 or 150 amino acids of the polypeptide of SEQ ID NO:14 are substituted with (a) conserved or non-conserved amino acid(s), which mutein lacks the N-terminal amino acids nos. 1-10, 1-9, 1-8, 1-7, 1-6, 1-5 or 1-4 of the mature SARP-1 polypeptide, and which mutein further has at least one of the biological activities of SARP-1.

The fusion protein according to the above embodiments may be glycosylated or non-glycosylated. Glycosylation of the fusion protein according to the above embodiments may have an impact on potential Fc mediated effector functions such as ADCC or CDC as it is known in the art.

In a further embodiment the fusion protein according to any of the above embodiments comprises at least 95%, 90%, 85%, 80%, 75%, 70%, 65%, 60% or 55% hybrid non-fucosylated bisected glycans.

In a further embodiment the fusion protein according to any of the above embodiments of the invention comprises only high mannose oligosaccharides.

In a further embodiment the fusion protein, variant or mutein according to any of the above embodiments has an essentially unaltered or reduced immunogenicity in humans as compared to the human SARP-1 polypeptide.

Another embodiment of the invention is a polynucleotide encoding a fusion protein according to any of the above embodiments.

Another embodiment of the invention is polynucleotide encoding a fusion protein comprising a mature SARP-1 polypeptide without the Netrin domain, and the fusion protein further comprising the Fc region of an immunoglobulin heavy chain, wherein the fusion protein is characterized in that it lacks the N-terminal amino acids nos. 1-10, 1-9, 1-8, 1-7, 1-6, 1-5 or 1-4 of the mature SARP-1 polypeptide.

Another embodiment of the invention is polynucleotide encoding a fusion protein comprising amino acids nos. 35 to 153 of SEQ ID NO: 6, 8 or 10, or comprising amino acids nos. 35 to 151 of SEQ ID NO:4, and the fusion protein further comprising the Fc region of an immunoglobulin heavy chain, such as an IgG₁, IgG₂, IgG₃ or IgG₄ Fc region of SEQ ID NOs:15, 16, 17 and 18, respectively, or any IgG₁, IgG₂, IgG₃ or IgG₄ Fc region with an amino acid sequence which is at least 99%, 98%, 97%, 96%, 95%, 94%, 93%, 92%, 91%, 90%, 85% or 80% A identical to the amino acid sequence of SEQ ID NO:15, 16, 17 or 18.

Another embodiment of the invention is a vector comprising a polynucleotide according to any of the above embodiments of the invention.

Another embodiment of the invention is a vector comprising a polynucleotide according to any of the above embodiments of the invention and the coding sequence for the mIgSP-tPA-pro signal peptide according to SEQ ID NO:24.

Another embodiment of the invention is a host cell comprising a polynucleotide, or a vector according to any of the above embodiments of the invention.

Another embodiment of the invention is a process for preparing a fusion protein according to any of the above embodiments of the invention, comprising expressing said fusion protein in a host cell according to the above embodiment of the invention, and recovering said fusion protein.

Another embodiment of the invention is a process for preparing a fusion protein according to any of the above embodiments of the invention, comprising expressing said fusion protein in a host cell according to the above embodiment of the invention, and isolating said fusion protein from the host cells or the host cell culture supernatant.

Another embodiment of the invention is the fusion protein according to any of the above embodiments for use as a medicament.

Another embodiment of the invention is a fusion protein according to any of the above embodiments for the treatment of cancer, a fibrotic disorder or a cardiovascular disorder.

Another embodiment of the invention is a fusion protein according to any of the above embodiments for the treatment of cancer, wherein the cancer is a gastrointestinal cancer, colorectal cancer, bladder cancer, pancreatic cancer, endometrial cancer, ovarian cancer, melanoma, leukemia and non-Hodgkin lymphoma, breast cancer, prostate cancer, or lung cancer.

Another embodiment of the invention is a fusion protein according to any of the above embodiments for the treatment of cancer, wherein the cancer is acute lymphoblastic leukemia, acute myeloid leukemia, adult, acute myeloid leukemia, adrenocortical carcinoma, astrocytoma, basal cell carcinoma, bile duct cancer, bladder cancer, bone cancer, such as osteosarcoma and malignant fibrous histiocytoma, glioma, ependymoma, medulloblastoma, breast cancer, bronchial adenomas, cervical cancer, chronic lymphocytic leukemia, chronic myelogenous leukemia, colon cancer, colorectal cancer, endometrial cancer, esophageal cancer, Ewing's family of tumors, extracranial germ cell tumor, extragonadal germ cell tumor, extrahepatic bile duct cancer, intraocular melanoma, retinoblastoma, gallbladder cancer, gastric (stomach) cancer, gastrointestinal carcinoid tumor, gastrointestinal stromal tumor (GIST), germ cell tumor, gestational trophoblastic tumor, hairy cell leukemia, head and neck cancer, hepatocellular (liver) cancer, lymphoma, such as Hodgkin's lymphoma, non-Hodgkin's lymphoma or Burkitt's lymphoma, cutaneous T-cell lymphoma, such as mycosis fungoides and Sézary syndrome, hypopharyngeal cancer, melanoma, such as intraocular melanoma, islet cell carcinoma (endocrine pancreas), Kaposi's sarcoma, kidney (renal cell) cancer, laryngeal cancer, lip and oral cavity cancer, lung cancer, such as non-small cell lung cancer or small cell lung cancer, Waldenstrom's macroglobulinemia, Merkel cell carcinoma, mesothelioma, mouth cancer, multiple myeloma, myelodysplastic syndromes, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary tumor, plasma cell neoplasm, pleuropulmonary blastoma, prostate cancer, rectal cancer, rhabdomyosarcoma, salivary gland cancer, sarcoma, testicular cancer, throat cancer, thymoma, thyroid cancer, urethral cancer, or Wilms' tumor.

Another embodiment of the invention is a fusion protein according to any of the above embodiments for the treatment of a cardiovascular disorder, wherein the cardiovascular disorder is myocardial infarction, cardiomyopathy or hypertrophic cardiomyopathy.

Another embodiment of the invention is a fusion protein according to any of the above embodiments for the treatment of a fibrotic disorder, wherein the fibrotic disorder is scleroderma, unwanted or excessive scarring, lung fibrosis (for example without limitation idiopathic pulmonary fibrosis), liver fibrosis, fibrosis of the intestine, kidney fibrosis, heart fibrosis or skin fibrosis.

Another embodiment of the invention is a method of treating cancer, a fibrotic disorder or a cardiovascular disorder comprising administering to a person in need of treatment a therapeutically effective amount of a fusion protein according to the invention.

Another embodiment of the invention is a method of treating cancer comprising administering to a person in need of treatment a therapeutically effective amount of a fusion protein according to the invention, wherein the cancer is a gastrointestinal cancer, colorectal cancer, bladder cancer, pancreatic cancer, endometrial cancer, ovarian cancer, melanoma, leukemia and non-Hodgkin lymphoma, breast cancer, prostate cancer, or lung cancer.

Another embodiment of the invention is a method of treating a cardiovascular disorder comprising administering to a person in need of treatment a therapeutically effective amount of a fusion protein according to the invention, wherein the cardiovascular disorder is myocardial infarction, cardiomyopathy or hypertrophic cardiomyopathy.

Another embodiment of the invention is a method of treating a fibrotic disorder comprising administering to a person in need of treatment a therapeutically effective amount of a fusion protein according to the invention, wherein the fibrotic disorder is scleroderma, unwanted or excessive scarring, lung fibrosis (for example without limitation idiopathic pulmonary fibrosis), liver fibrosis, fibrosis of the intestine, kidney fibrosis, heart fibrosis or skin fibrosis.

Another embodiment of the invention is a pharmaceutical composition comprising a fusion protein according to any of the above embodiments as active ingredient, optionally together with a pharmaceutically acceptable carrier or excipient.

The term “SARP-1”, “secreted apoptosis-related protein 1”, “sFRP-2”, “sFRP2”, or “secreted Frizzled-related protein 2” as used herein refers to any of the polypeptides of SEQ ID NO:1, 4, 6, 8 or 10.

The term “Netrin domain” of SARP-1 refers to a characteristic protein domain as it is known in the art, and which corresponds to the region from about amino acid 172 to about amino acid 295 of SEQ ID NOs:6, 8 and 10.

The term “a mature SARP-1 polypeptide” or “the mature SARP-1 polypeptide” as used herein refers to SARP-1 as shown in SEQ ID NO:1, 4, 6, 8 or 10 without the signal peptide. The signal peptide corresponds to the first 24 amino acids of SEQ ID NO:1, 4, 6, 8 or 10. An example of a “mature SARP-1 polypeptide” is also shown in SEQ ID NO:2.

The term “SARP-1(Fz)deltaN-Fc” as used herein refers to a fusion protein comprising a mature SARP-1 polypeptide without the Netrin domain, and the fusion protein comprising an Fc region of an immunoglobulin heavy chain heavy chain, wherein the fusion protein is characterized in that a defined number of N amino acids at the N-terminal amino acids of mature SARP-1 are lacking, wherein N=4, 5, 6, 7, 8, 9 or 10. For example without limitation, “SARP-1(Fz)delta7-Fc” corresponds to said protein wherein the first seven N-terminal amino acids are lacking; i.e. to a polypeptide comprising amino acids nos. 32 to 153 of SEQ ID NO: 6, 8 or 10, or comprising amino acids nos. 32 to 151 of SEQ ID NO:4.

The term “variants of SARP-1(Fz)deltaN-Fc” as used herein relates to polypeptides that have at least 99%, 98%, 97%, 96%, 95%, 94%, 93%, 92%, 91%, 90%, 85%, 80%, 75% or 70% identity with the polypeptide of SEQ ID NO:14, lack the N-terminal amino acids nos. 1-10, 1-9, 1-8, 1-7, 1-6, 1-5 or 1-4 of the mature SARP-1 polypeptide, and further have at least one of the biological activities of SARP-1. Biological activities of SARP-1 are known in the art and are also described herein. Biological activities of SARP-1 are for example but without limitation binding to Wnt1, Wnt4, Wnt 7a, Wnt 8 or Wnt9; antagonizing Wnt1, Wnt4, Wnt 7a, Wnt 8 or Wnt9 or reduction of lung fibrosis in the bleomycin-induced lung fibrosis mouse model as known in the art. A biological assay for determining Wnt antagonistic activity of SARP-1 is described in Example 4 herein. A biological assay for determining the inhibition by SARP-1 of PDGF-induced DNA synthesis in primary human hepatic stellate cells is described in Example 5. A biological assay for determining the inhibition by SARP-1 of the proliferation of cancer cells is described in Example 6. A biological assay for determining the inhibition by SARP-1 of renal fibrosis in a murine model is described in Example 7. A biological assay for determining the inhibition by SARP-1 of lung fibrosis in a murine model is described in Example 8.

The term “derivative(s) of SARP-1(Fz)deltaN-Fc” or “functional derivative(s) of SARP-1(Fz)deltaN-Fc” as used herein, relate to polypeptides that are derived from of SARP-1(Fz)deltaN-Fc, lack the N-terminal amino acids nos. 1-10, 1-9, 1-8, 1-7, 1-6, 1-5 or 1-4 of the mature SARP-1 polypeptide, and have at least one of the biological activities of SARP-1. Such “derivatives of SARP-1(Fz)deltaN-Fc” or “functional derivatives of SARP-1(Fz)deltaN-Fc” include for example, without limitation, esters or aliphatic amides of the carboxyl-groups and N-acyl derivatives of free amino groups or O-acyl derivatives of free hydroxyl-groups and are formed with acyl-groups as for example alcanoyl- or aroyl-groups. Alternatively, the derivatives may contain sugars or phosphates groups linked to the functional groups present on the lateral chains of the amino acid moieties. The derivatives may also comprise polyethylene glycol side chains. Such molecules can result from in vivo or in vitro processes which do not normally alter primary sequence, for example chemical derivatization of peptides (acetylation or carboxylation), phosphorylation (introduction of phosphotyrosine, phosphoserine, or phosphothreonine residues) or glycosylation (by exposing the peptide to enzymes which affect glycosylation e.g., mammalian glycosylating or deglycosylating enzymes).

The term “muteins of SARP-1(Fz)deltaN-Fc” refers to SARP-1(Fz)deltaN-Fc polypeptides in which one or more of the amino acid residues are substituted with a conserved or non-conserved amino acid residue (preferably a conserved amino acid residue) and such substituted amino acid residue may or may not be one encoded by the genetic code, wherein the muteins of SARP-1(Fz)deltaN-Fc lack the N-terminal amino acids nos. 1-10, 1-9, 1-8, 1-7, 1-6, 1-5 or 1-4 of the mature SARP-1 polypeptide, and further have at least one of the biological activities of SARP-1. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such muteins also include polypeptides in which one or more of the amino acid residues are substituted. In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule. Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs. The groups of synonymous amino acids and the groups of more preferred synonymous amino acids are shown in Table 1.

TABLE 1

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

The term “Fc fragment” as used herein refers to a dimer comprising two immunoglobulin heavy chains that each lack the variable (V) domain and the first constant (CH₁) domain, and optionally part of or the complete hinge region. An Fc fragment can be generated from a tetrameric immunoglobulin (the immunoglobulin comprising two heavy and two light chains) through papain digestion, as it is known in the art.

The term “Fc region” as used herein refers to a single immunoglobulin heavy chains that lacks the V domain and the CH₁ domain, and optionally part of or the complete hinge region. Two Fc regions form an Fc fragment.

The term “vector” refers to any polynucleotide that is useful for transferring exogenous DNA to a host cell for replication and/or appropriate expression of the exogenous DNA by the host cell, such as for example without limitation plasmids, expression vectors, viral vectors etc.

The host cells and vectors according to the above embodiments of the invention and that find use in the process for preparing a fusion protein according to any of the above embodiment of the invention are known in the art. The vector sequences may comprise further elements serving for expression of the polynucleotide of the invention. They may comprise regulatory sequence, such as promoter and enhancer sequences, selection marker sequences, origins of multiplication, and the like.

The vectors according to the embodiments of the invention may allow the expression of the fusion protein of the invention not only in the condition of tissue culture but also in vivo, for either experimental or therapeutic reasons. For example without limitation, cells over-expressing the fusion protein according to the embodiments of the invention can be transferred in an animal model to check the physiological effects of the constant administration of the fusion protein, and eventually before applying the cells to humans. Alternatively, the vector can be used for retrovirus-mediated gene transfer, or any other technology allowing the introduction and the expression of a vector or of the isolated DNA coding sequence in animal under the control of an endogenous promoter. This approach allows the generation of transgenic non-human animals in which the fusion protein according to the embodiments of the invention are expressed constitutively or in a regulated manner (for example without limitation in specific tissues and/or following the induction with specific compounds).

In general, the vectors can be episomal or non-/homologously integrating vectors, which can be introduced in the appropriate host cells by any suitable means (transformation, transfection, conjugation, protoplast fusion, electroporation, calcium phosphate-precipitation, direct microinjection, etc.) to transform them. The vectors should allow the expression of the fusion protein of the invention comprising them in the prokaryotic or eukaryotic host cell under the control of appropriate transcriptional initiation/termination regulatory sequences, which are chosen to be constitutively active or inducible in said cell. A cell line substantially enriched in such cells can be then isolated to provide a stable cell line.

Host cells according to the embodiments of the invention are for example bacterial, yeast (for example without limitation Candida boidinii, Hansenula polymorpha, Pichia methanolica, Pichia pastoris, Saccharomyces cerevisiae, Schizosaccharomyces pombe, Kluyveromyces lactis and other Kluyveromyces spp., Yarrowia lipolytica), Myxomycete (for example without limitation Dictyostelium discoideum), filamentous fungi (for example without limitation Trichoderma reesei and other Trichoderma spp., Aspergillus niger and other Aspergillus spp.), moss (for example without limitation Physcomitrella patens, Atrichum undulatum), insect or mammalian cells. Mammalian host cells are, for example without limitation of NS0, SP2.0, 3T3 cells, COS cells, human osteosarcoma cells, MRC-5 cells, baby hamster kidney (BHK) cells, VERO cells, CHO cells, rCHO-tPA cells, rCHO-Hep B Surface Antigen cells, CHO-S cells, HEK 293 cells, rHEK 293 cells, C127 cells, rC127-Hep B Surface Antigen cells, human fibroblast cells, Stroma cells, hepatocyte cells or PER.C6 cells.

For eukaryotic host cells (e.g. yeasts, insect or mammalian cells), different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells, which have been stably transformed by the introduced DNA, can be selected by also introducing one or more markers, which allow for selection of host cells, which contain the expression vector. The marker may also provide for phototrophy to an auxotropic host, biocide resistance, for example without limitation antibiotics, or heavy metals such as copper, or the like. The selectable marker gene can either be directly linked to the DNA gene sequences to be expressed, or introduced into the same cell by co-transfection. Additional elements may also be needed for optimal synthesis of proteins of the invention.

The process for preparing a fusion protein according to any of the above embodiments of the invention recombinant expression may employ eukaryotic expression systems, such as insect cells, and mammalian expression systems because they provide post-translational modifications to protein molecules, including correct folding or glycosylation at correct sites. Alternative eukaryotic host cells are yeast cells transformed with yeast expression vectors. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast recognizes leader sequences in cloned mammalian gene products and secretes peptides bearing leader sequences (i.e., pre-peptides).

The fusion protein according to the embodiments of the invention can also be produced in transgenic plants such as rice, potato, tobacco, clover, canola, corn, barley, wheat, maize, soybean, cassaya, alfalfa, banana, carrots, tomato or legume such as Medicago truncatula as it is described in the art (Nikolov and Woodard, 2004; Hellwig et al., 2004). For example, numerous recombinant immunoglobulins or immunoglobulin fragments have been produced in transgenic plants (Fischer et al., 2003; Goldstein and Thomas, 2004).

For long-term, high-yield production of a fusion protein according to the invention, stable expression is preferred. For example without limitation, cell lines, which stably express a polypeptide of interest, may be transformed using expression vectors, which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type. A cell line substantially enriched in such cells can be then isolated to provide a stable cell line.

A particularly preferred process for preparing a fusion protein according to any of the embodiments employs dihydrofolate reductase (DHFR) amplification in DHFR-deficient cells, e.g. DHFR-deficient CHO cells, by the use of successively increasing levels of methotrexate as known in the art.

For sequences which are not identical, a “% identity” may be determined. “% identity” as used herein refers to the identity of sequence A to sequence B over the whole length of sequence A. In general, the two sequences to be compared are aligned to give a maximum correlation between the sequences. This may include inserting “gaps” in either one or both sequences, to enhance the degree of alignment. Methods for determining the identity of two sequences are well known in the art. A preferred way to determine the % identity between two polynucleotide sequences or the % identity between two polypeptide sequences uses the BLAST 2 Sequences software (Tatusova and Madden, 1999), which is available e.g. through the National Center for Biotechnology Information, National Library of Medicine, National Institutes of Health, Building 38A, 8600 Rockville Pike, Bethesda, Md. 20894, USA (Wheeler et al., 2007; Pearson, 1990a; Pearson, 1990b).

A simple way to calculate the % identity of a polypeptide A with a polypeptide B, is to align the two polypeptide sequences A and B, for example without limitation by using any of the algorithms known in the art, such as the BLAST 2 Sequences algorithm, or manually by matching the highest possible number of identical amino acids, and calculate how many amino acids of polypeptide A match an identical amino acid in polypeptide B. The number of matching amino acids is then set in relation to the overall number of amino acids of the polypeptide A, which provides the value for the % identity of polypeptide A to polypeptide B. The % identity of polynucleotide A with polynucleotide B is calculated in an analogous manner.

The term “polynucleotide” or “polynucleotides” relates to RNA, DNA, cDNA, or analogues thereof including, but not limited to, locked nucleic acid (LNA), peptide nucleic acid (PNA), morpholino nucleic acid, glycol nucleic acid (GNA) and threose nucleic acid (TNA).

The terms “treating” and “preventing”, as used herein, should be understood as preventing, inhibiting, attenuating, ameliorating or reversing one or more symptoms or cause(s) of disease, as well as symptoms, diseases or complications accompanying disease. When “treating” disease, the fusion protein or pharmaceutical compositions according to the invention are given after diagnosis or onset of the disease, “prevention” relates to administration of the substances before any pathological changes or symptoms of the disease to be prevented can be noted by any means in the individual.

The term “cancer”, as used herein refers class of diseases or disorders characterized by uncontrolled division of cells and the ability of these to spread, either by direct growth into adjacent tissue through invasion, or by implantation into distant sites by metastasis. The following closely related terms are used to designate uncontrolled division of cells a mentioned above: Neoplasia and neoplasm are the scientific designations for cancerous diseases. This group contains a large number of different diseases. Neoplasms can be benign or malignant. Cancer is a widely used word that is usually understood as synonymous with malignant neoplasm. It is occasionally used instead of carcinoma, a sub-group of malignant neoplasms. Tumor in medical language simply means swelling or lump, either neoplastic, inflammatory or other. In common language, however, it is synonymous with ‘neoplasm’, either benign or malignant.

Cancers are classified by the type of cell that resembles the tumor and, therefore, the tissue presumed to be the origin of the tumor. The following general categories are usually accepted: Carcinoma: malignant tumors derived from epithelial cells. This group represents the most common cancers, including the common forms of breast, prostate, lung and colon cancer. Lymphoma and Leukemia: malignant tumors derived from blood and bone marrow cells. Sarcoma: malignant tumors derived from connective tissue, or mesenchymal cells. Mesothelioma: tumors derived from the mesothelial cells lining the peritoneum and the pleura. Glioma: tumors derived from glia, the most common type of brain cell. Germinoma: tumors derived from germ cells, normally found in the testicle and ovary. Choriocarcinoma: malignant tumors derived from the placenta.

The most common types of cancer are prostate cancer, lung cancer, breast cancer, colorectal cancer, bladder cancer, pancreatic cancer, endometrial cancer, ovarian cancer, melanoma, leukemia and non-Hodgkin lymphoma.

Other cancers are acute lymphoblastic leukemia, acute myeloid leukemia, adult, acute myeloid leukemia, adrenocortical carcinoma, astrocytoma, basal cell carcinoma, bile duct cancer, bladder cancer, bone cancer, such as osteosarcoma and malignant fibrous histiocytoma, glioma, ependymoma, medulloblastoma, breast cancer, bronchial adenomas, cervical cancer, chronic lymphocytic leukemia, chronic myelogenous leukemia, colon cancer, colorectal cancer, endometrial cancer, esophageal cancer, Ewing's family of tumors, extracranial germ cell tumor, extragonadal germ cell tumor, extrahepatic bile duct cancer, intraocular melanoma, retinoblastoma, gallbladder cancer, gastric (stomach) cancer, gastrointestinal carcinoid tumor, gastrointestinal stromal tumor (GIST), germ cell tumor, gestational trophoblastic tumor, hairy cell leukemia, head and neck cancer, hepatocellular (liver) cancer, lymphoma, such as Hodgkin's lymphoma, non-Hodgkin's lymphoma or Burkitt's lymphoma, cutaneous T-cell lymphoma, such as mycosis fungoides and Sézary syndrome, hypopharyngeal cancer, melanoma, such as intraocular melanoma, islet cell carcinoma (endocrine pancreas), Kaposi's sarcoma, kidney (renal cell) cancer, laryngeal cancer, lip and oral cavity cancer, lung cancer, such as non-small cell lung cancer or small cell lung cancer, Waldenstrom's macroglobulinemia, Merkel cell carcinoma, mesothelioma, mouth cancer, multiple myeloma, myelodysplastic syndromes, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary tumor, plasma cell neoplasm, pleuropulmonary blastoma, prostate cancer, rectal cancer, rhabdomyosarcoma, salivary gland cancer, sarcoma, testicular cancer, throat cancer, thymoma, thyroid cancer, urethral cancer, and Wilms' tumor.

The term “fibrotic disorder”, as used herein refers to a disorder characterized by the formation or development of excess fibrous connective tissue in an organ or tissue, frequently as a reparative or reactive process. Fibrosis can affect single organs, such as the lungs (for example without limitation idiopathic pulmonary fibrosis), the liver, the intestine, the kidney, the heart or the skin, or affect multiple organs, for example without limitation in systemic sclerosis. The term fibrotic disorder also relates to scarring of the skin. Scars of the skin include, but are not limited to, keloid scars, contracture scars that occur, for example without limitation after skin burn, hypertrophic scars and acne scars.

The term “cardiovascular disorder”, as used herein refers to a group of diseases that affect the heart or the blood vessels. “Cardiovascular disorder” includes, but is not limited to, aneurysma; angina; arrhythmia; atherosclerosis; cardiomyopathy; congenital heart disease; congestive heart failure; myocarditis; valve disease; coronary artery disease; dilated cardiomyopathy; diastolic dysfunction; endocarditis; hypertension; hypotension; hypertrophic cardiomyopathy; mitral valve prolapse; myocardial infarction; stroke and venous thromboembolism.

The definition of “pharmaceutically acceptable” is meant to encompass any carrier, which does not interfere with effectiveness of the biological activity of the active ingredient and that is not toxic to the host to which it is administered. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil). For example without limitation, for parenteral administration, the above active ingredients may be formulated in unit dosage form for injection in vehicles such as saline, dextrose solution, serum albumin and Ringer's solution.

The pharmaceutical composition according to the invention can be administered to an individual systemically or locally. Systemic administration is, for example without limitation, achieved by administration through the digestive tract (enteral administration) or through other routes (parenteral administration). Parenteral administration routes are, for example without limitation, intravenous, intraarterial, subcutaneous, transdermal, intradermal, intramuscular, intraperitoneal, nasal, intracranial, intrathecal, intracardiac, intraosseous or transmucosal routes. Enteral administration routes are, for example without limitation, oral, rectal, sublingual, or buccal routes. Local administration is achieved, for example without limitation, through topical, epidural, epicutaneous, inhalational, nasal, intraarticular, vaginal, auricular or intravitreal routes.

The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example without limitation, sesame oil, or synthetic fatty acid esters, for example without limitation, sesame oil, or synthetic fatty acid esters, for example without limitation, ethyl oleate or triglycerides. Aqueous injection suspensions that may contain substances increasing the viscosity of the suspension include, for example without limitation, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The optimal dose of the pharmaceutical composition may be appropriately selected according to the route of administration, patient conditions and characteristics (sex, age, body weight, health, size), extent of symptoms, concurrent treatments, frequency of treatment and the effect desired. Adjustment and manipulation of established dosage ranges are well within the ability of those skilled. Any other therapeutically efficacious route of administration can be used, for example absorption through epithelial or endothelial tissues. In addition, the protein(s) according to the invention can be administered together with other components of biologically active agents such as pharmaceutically acceptable surfactants, excipients, carriers, diluents and vehicles.

For parenteral (for example without limitation intravenous, subcutaneous, intramuscular) administration, the active ingredient can be formulated as a solution, suspension, emulsion or lyophilized powder in association with a pharmaceutically acceptable parenteral vehicle (for example without limitation water, saline, dextrose solution) and additives that maintain isotonicity (e.g. mannitol) or chemical stability (e.g. preservatives and buffers). The formulation is sterilized by commonly used techniques.

The therapeutically effective amounts of the active ingredient will be a function of many variables, including but without limitation, the route of administration, the clinical condition of the patient, the pharmacokinetics of the active ingredient in a patient.

A “therapeutically effective amount” is amount of a fusion protein according to any of the embodiments of the invention that when administered to a patient in need of treatment with said fusion protein, such as a patient suffering from a from cancer, a fibrotic disorder or a cardiovascular disorder, the amount of said fusion protein results in an improvement of the disorder un that patient vis-à-vis a patient who did not receive a therapeutically effective amount of said fusion protein. An improvement of the disorder can be measured by methods known in the art, the methods including the measurement of laboratory parameters taken from blood, urine, synovial fluid or cerebrospinal fluid, or other body fluids, the measurement of the functional status, pain or disability; the methods also including imaging such as magnetic resonance imaging (MRI) or X-ray.

The dosage administered, as single or multiple doses, to an individual will vary depending upon a variety of factors, including pharmacokinetic properties of a fusion protein according to any of the embodiments of the invention, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the effect of said fusion protein in an individual.

A fusion protein according to any of the embodiments of the invention may be used in amounts in the ranges of 0.001 to 100 mg/kg or 0.01 to 10 mg/kg or body weight, or 0.1 to 5 mg/kg of body weight or 1 to 3 mg/kg of body weight or 2 mg/kg of body weight.

A fusion protein according to any of the embodiments of the invention may be administered daily or every other day or three times per week or once per week, every other week, once per month, every 6 weeks, every other month, 3 times per year, 2 times per year or once per year at similar doses, or at doses increasing or decreasing with the time.

The daily doses are usually given in divided doses or in sustained release form effective to obtain the desired results. Second or subsequent administrations can be performed at a dosage which is the same, less than or greater than the initial or previous dose administered to the individual. In a preferred embodiment the fusion protein according to any of the embodiments of the invention is administered at a first dose and one or more subsequent higher dose(s).

A fusion protein according to any of the embodiments of the invention may be administered prophylactically or therapeutically to an individual prior to, simultaneously or sequentially with other therapeutic regimens or agents (for example without limitation multiple drug regimens), in therapeutically effective amounts.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

As used herein, “a” or “an” may mean one or more. The use of the term “or” herein is used to mean “and/or” unless explicitly indicated to refer to alternatives only or the alternatives are mutually exclusive, although the disclosure supports a definition that refers to only alternatives and “and/or.” As used herein “another” may mean at least a second or more.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Example 1

### Analysis of the Signal Peptide of Human SARP-1

We analyzed the N-terminal parts and the region around the cleavage sites of sFRP2 (SARP-1) and its family homologues. Namely, we studied a number of the physicochemical properties of the signal peptide of sFRP2 in comparison with the corresponding sequence segments from the sequences of sFPR1 and sFRP3-5.

Notably, the five members of the sFRP family have distinct signal peptide lengths and sequence composition (see Table 2 below).

TABLE 2
Signal peptides of SARP polypeptides

| | | SP |
|---|---|---|
| | | length |
| Protein | Signal Peptide sequence\|<5aa> | (aa) |
| sFPR1_ | MGIGRSEGGRRGAALGVLLALGAALLAVGSA\| | 30 |
| HUMAN | SEYDY | |
| sFPR2_ | MLQGPGSLLLLFLASHCCLGSARG\|LFLFGQ | 24 |
| HUMAN | | |
| (SARP-1) | | |
| sFPR3_ | MVCGSPGGMLLLRAGLLALAALCLLRVPGARA\| | 31 |
| HUMAN | AACEP | |
| sFPR4_ | MFLSILVALCLWLHLALG\|VRGAP | 18 |
| HUMAN | | |
| sFPR5_ | MRAAAAAGGVRTAALALLLGALHWAPARC\| | 28 |
| HUMAN | EEYDY | |

We analysed the properties of hydrophilicity and surface accessibility of the sequence segments (using the Web-form based interface of the GCG package, PeptideStructure module), and we noted that, in large, they share a similar physicochemical profile. However, a part of the data obtained for sFRP2 were noteworthy different. The hydrophilicity profile for sFRP2 showed that the tetra-peptide (LFLF) following after the signal peptide introduces a highly hydrophobic motif into the N-terminus of sFRP2 (SARP-1). Comparison with the profiles of the other sFRP family members did not indicate such a bias in their N-terminal sequence composition. We predicted that this short very hydrophobic N-terminal segment would potentially have a rather flexible stretch of amino acids.

Based on these results we formed the hypothesis that the tetra-peptide LFLF interfered with cleavage of the signal peptide, and hence reduced the expression of the SARP-1(Fz)-Fc polypeptide. The hypothesis was tested experimentally and confirmed.

### Example 2

### Cloning of SARP-1(Fz)delta7-Fc

Cloning of SARP-1

Sequential BLAST searches were performed on the human NCBI dbEST starting with the partial coding sequence of SARP-1 (EMBL accession number: AF017986) and relevant ESTs were retrieved using ENTREZ at www.ncbi.nlm.gov/Web/Search/index.html. The following ESTs were then assembled along with the AF017986 sequence to generate the consensus full coding sequence of SARP-1: AW580647, AW608301, AA976403, and W92531. The full-length cDNA coding sequence of SARP-1 was then cloned by reverse transcriptase PCR from normal human dermal fibroblast RNA. The SARP-1 forward 5′ PCR primer contained HindIII and Kozak sequence (5′ GCC AAG CTT CCC ACG ATG CTG CAG GGC CCT). The SARP-1 reverse 3′ primer contains an XhoI site (5′GCG CTC GAG CTA GCA CTG CAG CTT GCG GAT). The PCR product was cleaved with the restriction enzymes HindIII and XhoI and cloned into a pcDNA3.1 vector after cleavage of the vector with the same restriction enzymes to generate SARP-1 plasmid.

Generation of SARP-1 (Fz-domain) Fc Fusion Construct [SARP-1(Fz)-Fc]

A clone of the pEAK12d expression vector containing a cDNA encoding a SARP-1 fragment comprising the signal peptide and the frizzled domain (the fragment from amino acids nos. 1-153 of SEQ ID NO:6) fused to a C-terminal Fc fragment (human IgG₁ heavy chain hinge region, CH₂ and CH₃ domains) was generated via a series of intermediate plasmids using the Gateway cloning technology (Invitrogen) as follows:

Gateway compatible cDNA containing the SARP-10RF flanked at the 5′ end by an attB1 recombination site and Kozak sequence (GCC ACC), and flanked at the 3′ end by a stop codon (TGA) and the attB2 recombination site was generated by two sequential PCR reactions. The Gateway modified PCR product was sub-cloned into the Gateway entry vector pDONR201 in a recombination reaction mediated by BP clonase (Invitrogen) to generate a Gateway entry clone pENTR-SARP-1. A SARP-1 Fc fusion construct was then made by overlapping PCR as follows: in the first PCR reaction, the SARP-10RF was amplified using the following PCR primers: Sarp1-B1p-121 (forward primer) 5′ GCA GGC TTC GCC ACC ATG CTG C and Sarp1-hFC-1036-R (reverse primer) 5′ CAC AAG ATT TGG GCT CGC ACT GCA GCT TGC GGA T. In a second PCR reaction, the cDNA sequence encoding the IgG₁ Fc domain was amplified. The PCR products from both reactions were combined, and a cDNA sequence encoding the full length SARP-1 Fc fusion protein [SARP-1-Fc] was generated in a third PCR reaction using a Gateway system universal forward primer, attB1-K, 5′ GGG GAC AAG TTT GTA CAA AAA AGC AGG CTT CGC CAC C and a Gateway system universal reverse primer, attB2, 5′ GGG GAC CAC TTT GTA CAA GAA AGC TGG GTT. The resultant PCR product (SARP-1 Fc fusion) was subcloned into pDONR 221 using the Gateway cloning technology (as described above), to generate hSARP-1-Fc.

The sequence encoding the Netrin domain SARP-1 (amino acids nos. 148 to 271 of SEQ ID NO:2) was then deleted by site directed mutagenesis using the Quick Change Site Directed Mutagenesis Kit (Stratagene) to create the plasmid for SARP-1(Fz)-Fc.

SARP-1(Fz)-Fc with the cognate signal peptide was only secreted at a rather low level, which was considered to be unsatisfactory. SARP-1(Fz)-Fc also exhibited an N-terminal heterogeneity when expressed in standard mammalian expression systems such as HEK and CHO. Therefore, and based on the results obtained according to Example 1, it was decided to delete the N-terminal amino acids, and furthermore an artificial signal peptide comprising the mouse immunoglobuline signal peptide (mIgSP) and the tissue plasminogen activator signal pro peptide (tPApro) was used for expression. Said artificial signal peptide (mIgSP-tPA-pro) is disclosed in WO 2005/030963.

To clone SARP-1(Fz)delta7-Fc, initially the SARP-1(Fz)-Fc fusion sequence was obtained by PCR amplification with 5′ tPA-SARP primer, reconstituting the 3′ part of the mIgSP-tPA-pro signal peptide with a EcoRI site, and 3′FcSARPwt primer. EcoRI and NheI restriction sites were integrated into the respective primers to facilitate cloning. The plasmid was cleaved by partial EcoRI digestion and then digested with NheI followed by incubation with calf intestinal alkaline phosphatase (CIAP) for 15 minutes. The PCR product was cleaved with EcoRI and NheI and subsequently cloned into the EcoRI (3388), cutting within the tPA part of the signal peptide, and NheI sites. The correct product was chosen after restriction analysis of the resulting SARP-1(Fz)delta7-Fc plasmid.

### Example 3

### Manufacture of SARP-1(Fz)delta7-Fc

CHO cells expressing SARP-1(Fz)delta7-Fc were seeded at 5×10⁵ cells/ml in 5 L of ProCHO5 medium (Lonza, Switzerland, BE12-762Q) supplemented with 4 mM glutamine and allowed to grow for 6 days. CHO culture supernatants were harvested and diluted 3/5 in 50 mM Tris-HCl pH 8.0 in order to adjust the pH to 7.5 and to lower the conductivity from 9.5 to around 6.5 mS/cm⁻¹. The solution was then filtered on an Acropak 1000-0.8/0.2 μm filter (PALL, Art. 406201010332). The solution was captured on a Q-Sepharose FF (GE-Healthcare, art. 17-0510-01) anion exchanger, run at a flux of 600 cm/hour on the resin pre-equilibrated with 0.1 M Tris-HCl pH 8.0. All fractions from the elution were collected, analysed by SDS-PAGE and Western blot for the presence of SARP-1(Fz)delta7-Fc. Q-Sepharose eluate was pooled, incubated in 0.5% Triton X-100 for 1 hour at room temperature (RT) and applied ton a affinity chromatography resin of Protein A coupled to agarose-beads (MabSelect™—Amersham). Elution from the resin was done with 0.1 M Na-Citrate pH 3.0 buffer. The eluate was collected into 0.1 M Tris-HCl pH 8.5 buffer resulting in pH adjustment to 7.6. The fractions containing SARP-1(Fz)delta7-Fc were pooled and dialysed against 1×PBS (2 changes) over 2 days, in Spectra/Por MWCO 6-8000 (Art. 132655) tubes. The protein was then stored in aliquots in cryovials at −80° C.

Results

N-terminal sequencing of the first 5 residues (Edman degradation) was performed on the purified protein SARP-1(Fz)delta7-Fc loaded on a ProSorb filter cartridge. All the batches produced had the N-terminal amino acid sequence DFSYK at 100% of material analyzed, as expected.

Amino acid analysis was performed to verify the purity of the protein and to verify the theoretical extinction coefficient for determination of the protein concentration. The recovery corresponded very well with the theoretical values, indicating a high purity of the protein. The protein concentrations determined by amino acid analysis of SARP-1(Fz)delta7-Fc was identical to that calculated by UV spectroscopy using the theoretical extinction coefficient of 50260 M⁻¹×cm⁻¹ suggesting a complete matching of the experimental and the theoretical extinction coefficients.

### Example 4

### Determination of Wnt Antagonistic Activity

Materials and Methods

293T cells expressing murine Wnt1 and human Wnt2 (Inducer Cells) and 293T cells expressing Luciferase under the control of LEF-site-linked promoter (Reporter Cells) were mixed at an equal ratio and seeded in 100 μl medium per well in a 96-well microtiter plate. The Inducer Cells by overexpressing Wnt1 and Wnt2 at its surface were able to induce an activation of the Wnt signaling pathway triggered by the interaction of the Wnt proteins with its receptor at the cell membrane of the Reporter Cells. The Reporter Cells in response to Wnt pathway activation expressed luciferase by the activation of Wnt responsive genes such as LEF.

The mixture was composed of about 10,000 Inducer Cells and 10,000 Reporter Cells per well. SARP-1(Fz)delta7-Fc was added to the culture medium at different concentrations (from 300 to 5 μg/ml). After 48 hours of incubation, the medium was discarded, and 40 μl lysis buffer were added to the cells. After 15 minutes of lysis on an orbital shaker at 4° C., the samples were submitted to two cycles of freezing and thawing followed by rigid shaking using a vortex (for 2 minutes) and sedimentation by centrifugation at 2000 rpm in V-shape microtiter plates.

Luciferase activities were normalized by total protein content in the lysate. Protein quantification was performed on 5 μl cell lysate using the BCA kit (Pierce) as known in the art. To measure the luciferase activity, 10 μl of cell lysate each was transferred into a 96-well microtiter plate. 15 μl of luciferin reagent was added to the sample and the quantification of the luminescence was counted during 10 seconds.

The results were expressed in percentage of Wnt activation. Thus, the maximal luciferase activity obtained with the mixture of Inducer and Reporter Cells corresponded to 100%.

Results

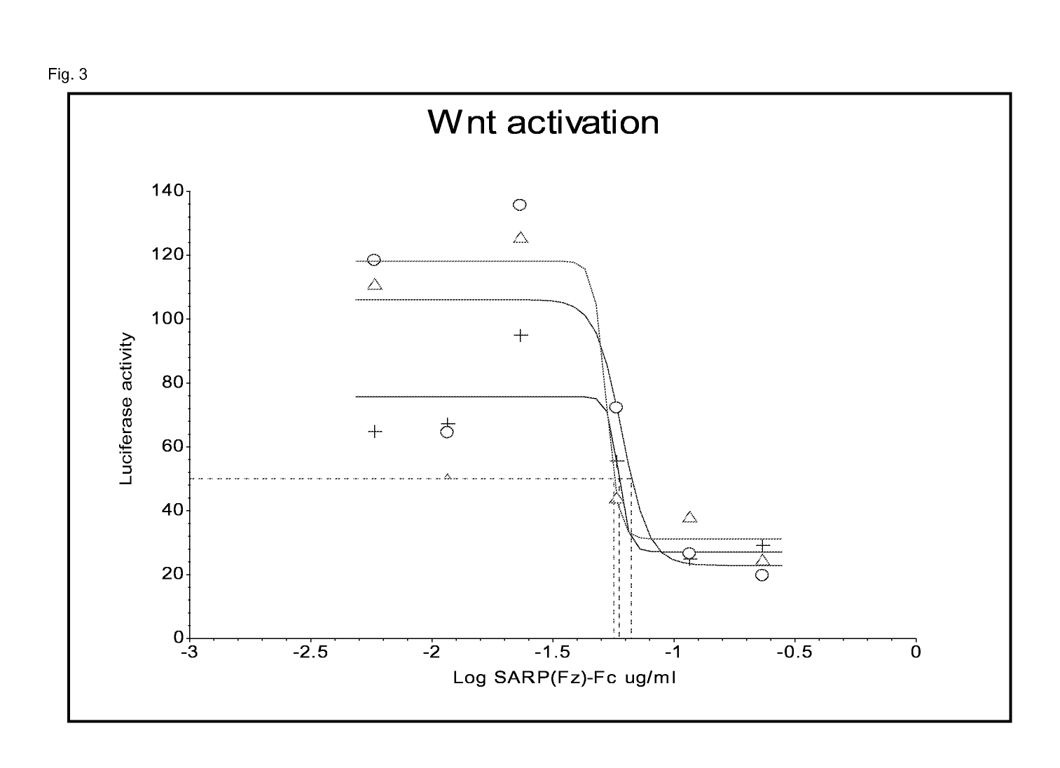

SARP-1(Fz)delta7-Fc reduced the luciferase activity in a dose dependent manner with an IC₅₀ of 0.75 μM. The maximum inhibition reached 80% at concentration from 150 to 300 μg/ml. This result are shown in FIG. 3 and demonstrate that SARP-1(Fz)delta7-Fc is able to block the induction of Wnt activation in a cellular system.

### Example 5

### Biological Effect on Primary Human Hepatic Stellate Cells

Materials and Methods

Human hepatic stellate cells (HSCs) were isolated from wedge sections of normal human liver unsuitable for transplantation. Liver tissue was digested with collagenase/pronase. HSC were separated from other liver nonparenchymal cells by ultracentrifugation over gradients of Stractan (Cellsep isotonic solution; Larex Inc., St. Paul, Minn.). HSC were cultured on plastic culture dishes in Iscove's modified Dulbecco's medium supplemented with 0.6 U/mL of insulin, 2.0 mmol/L of glutamine, 0.1 mmol/L of nonessential amino acids, 1.0 mmol/L of sodium pyruvate, antibiotic-antimycotic solution (all provided by Gibco Laboratories, Grand Island, N.Y.), and 20% fetal bovine serum (Imperial Laboratories, Andover, UK). All experiments were performed in triplicate and results are expressed as MEAN+SD. Statistical significance was evaluated by Student t test.

Effects on Cell Proliferation

For this group of experiments the stimulus of choice was human recombinant PDGF-BB at the standard dose of 10 ng/ml. Canrenone (10 μM), whose inhibitory effect has been previously shown in the same cell preparations (Caligiuri et al., 2003), was used as a positive control of inhibition.

1. Cell Proliferation Assay

HSC were plated in 12 well dishes at 30% confluence in Iscove's modified Dulbecco's medium supplemented with 0.6 U/mL insulin, 2.0 mmol/L of glutamine, 0.1 mmol/L nonessential amino acids, 1.0 mmol/L of sodium pyruvate, antibiotic-antimycotic solution (all from Gibco Laboratories, Grand Island, N.Y.), and 20% fetal bovine serum (Imperial Laboratories, Andover, UK). After 24 hours the HSC were incubated with serum-free medium containing PDGF-BB to induce activation at a concentration of 10 ng/ml with or without SARP-1(Fz)delta7-Fc at a concentration of 10, 1 and 0.1 μg/ml. HSC were harvested and cell number/well were determined after 2, 4, and 6 days of culture. At each time point fresh medium and experimental conditions were added at the remaining wells.

2. Incorporation of 3^{H}-Thymidine into DNA

HSC were plated in 24 well plastic dishes and grown to confluence in complete cell culture medium containing 20% of fetal bovine serum (FBS). HSC were then deprived of serum for 48 hours and stimulated with PDGF-BB for additional 24 hours. 3″-thymidine is added during the last 4 hours of incubation. SARP-1(Fz)delta7-Fc was added immediately before PDGF at a concentration of 10, 1 and 0.1 μg/ml.

Results

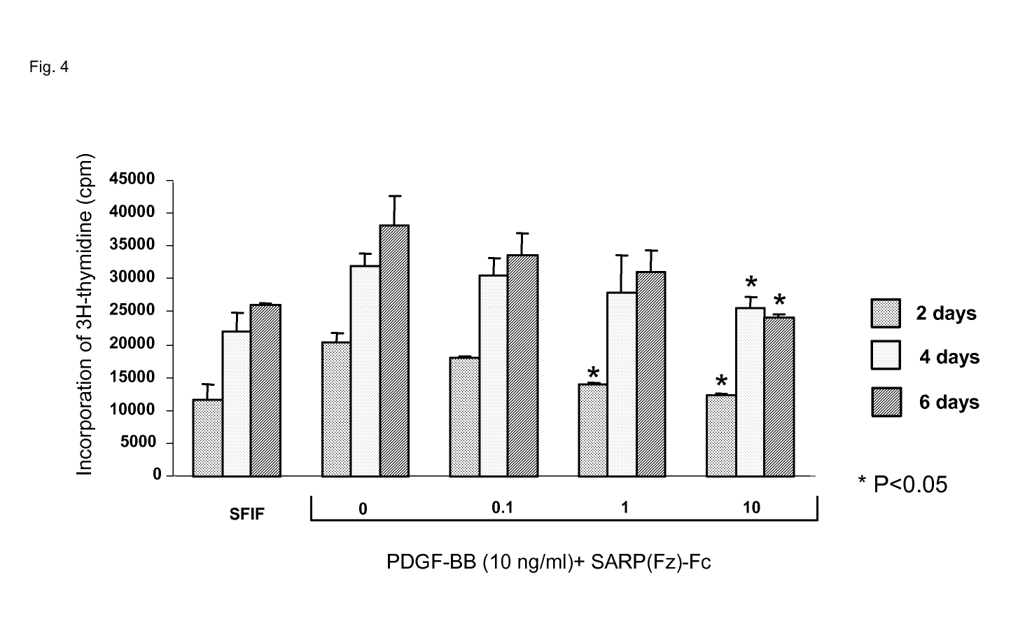

SARP-1(Fz)delta7-Fc reduced significantly PDGF-induced DNA synthesis in the primary human hepatic stellate cells cultures. This inhibitory effect was statistically significant starting at dose of 1 μg/ml after 2 days of culture and more prominent at 10.0 μg/ml after 2, 4 and 6 days of culture. The results are shown in FIG. 4 and demonstrate that recombinant SARP-1(Fz)delta7-Fc is able to reduce the proliferation of primary human hepatic stellate cells. This effect indicates a beneficial effect in the treatment of liver fibrosis.

### Example 6

### Effect of SARP-1(Fz)delta7-Fc on Human Cancer Cells

Materials and Methods

The cell line B16F1 (mouse melanoma) were cultivated in DMEM+10% serum and seeded at 5000 cells/well in a total volume of 50 μl. The SW480 and SW620 (human colon carcinoma) cell lines were cultivated in L15 medium+10% fetal bovine serum and seeded at 20,000 cells per well in a total volume of 50 μl. SARP-1(Fz)delta7-Fc was added immediately at a concentration of 1, 3 or 6 μM.

Cell proliferation assay was evaluated by reduction of 3-(4,5-dimethylthiazol-2-yl)-2,5-diphenyltetrazolium bromide (MTT) after 24, 48, 72 and 96 hours of culture. For the test of cell proliferation, 5 μl and 10 μl of MTT solution were added to the cultures of B16F1, SW480 and SW620 cells, respectively. After the incubation period, cells were lysed in 200 μl DMSO, and optic density was determined at 570 and 630 nm. The positive control for inhibition of cell proliferation was DKK1-Fc used at a concentration of 1 μM during the experiment.

Results

A significant inhibition of proliferation of B16F1 melanoma cell line was obtained with SARP-1(Fz)delta7-Fc at concentrations from 1 to 6 μM with a dose-response effect. The level of inhibition obtained with 6 μM of SARP-1(Fz)delta7-Fc is comparable to the one obtained by using the positive control (DKK1-Fc).

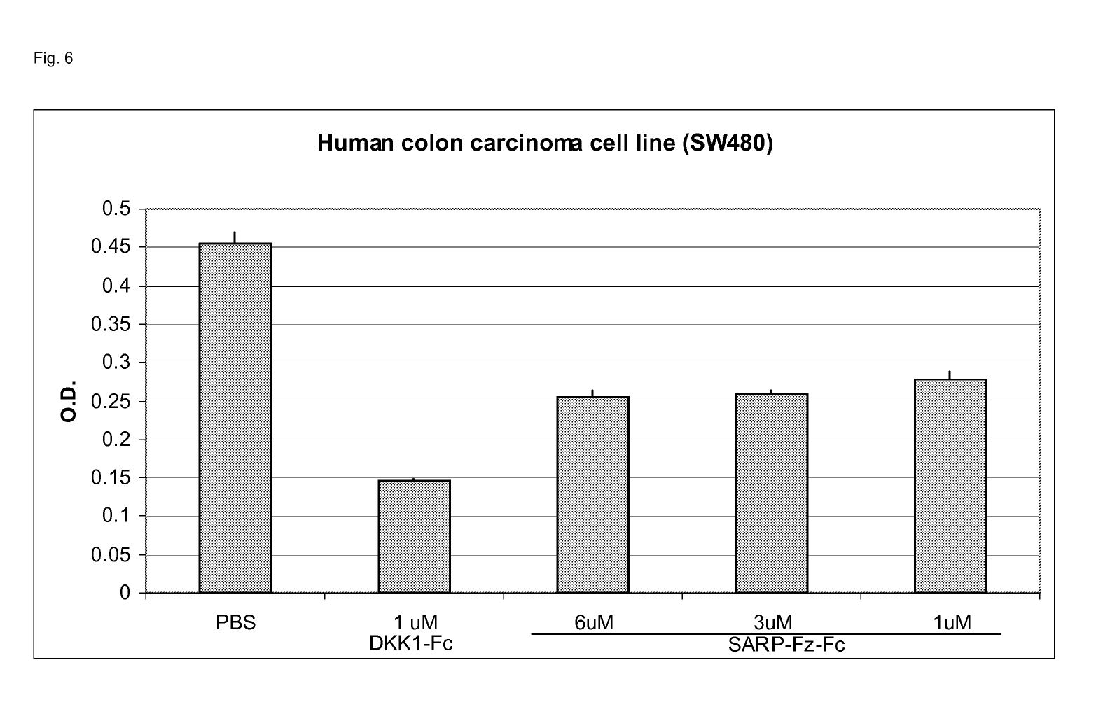

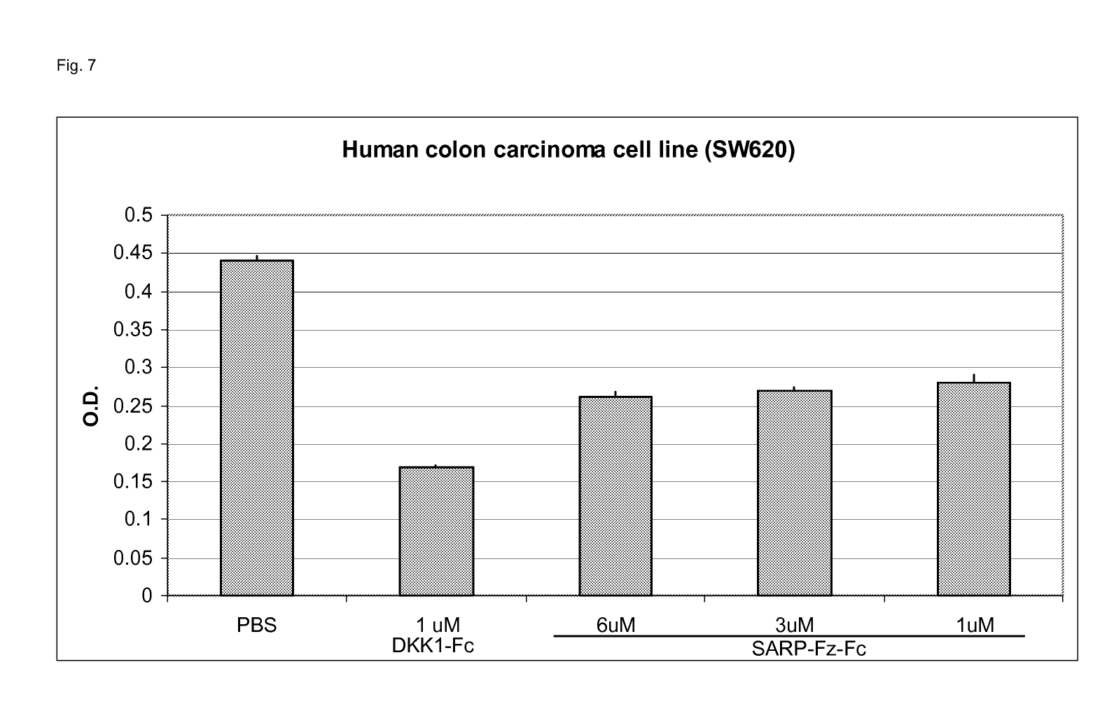

Similarly, SARP-1(Fz)delta7-Fc was able to inhibit the cell proliferation of the two human colon carcinoma cell lines SW480 and SW620. The results are shown in FIGS. 6, 7 and 8.

This effect indicates a potential benefic effect of SARP-1(Fz)delta7-Fc in the treatment of cancer.

### Example 7

### In Vivo Activity of SARP-1(Fz)delta7-Fc in a Murine Model of Renal Fibrosis

Materials and Methods

The in vivo activity of SARP-1(Fz)delta7-Fc was tested in an experimental model of obstructive nephropathy, which is unilateral ureter ligation-induced renal fibrosis (Vielhauer et al., 2001).

Female inbred C57BL/6 mice weighing 20 to 26 g were kept in macrolone type III cages under a 12 h light/dark cycle. Water and standard chow were available ad libitum. Under general ether anesthesia, a low midline abdominal incision was performed and the ligation of the left distal ureter was made with a 2/0 Mersilene suture, resulting in a unilateral ureter obstruction (UUO). Unobstructed contralateral kidneys served as controls.

Subsequently, mice were treated subcutaneously with 1.5 mg/kg SARP-1(Fz)delta7-Fc in 50 μl vehicle (0.9% NaCl) or vehicle alone. The first dose was administered immediately after UUO once a day for 21 days

Mice were killed at 7 and 21 days after UUO by cervical dislocation under general anesthesia with inhaled ether.

Analysis of Renal Morphology and Immunohistochemistry

From each mouse cranial kidney halves were used for histological assessment. Ligated and contralateral kidney tissue was fixed for 24 hours at room temperature in 4% neutral buffered formalin and then embedded in paraffin. For quantitative analysis, 4 μm horizontal sections were cut. Every fifth of fifteen subsequent sections, chosen by systematic uniformly random sampling, was used for analysis. Slides were stained with periodic acid-Schiff (PAS) reagent for routine histology and morphometric analysis and with anti-α-SMA (α-smooth muscle actin) to evaluate the presence of myofibroblast.

Results

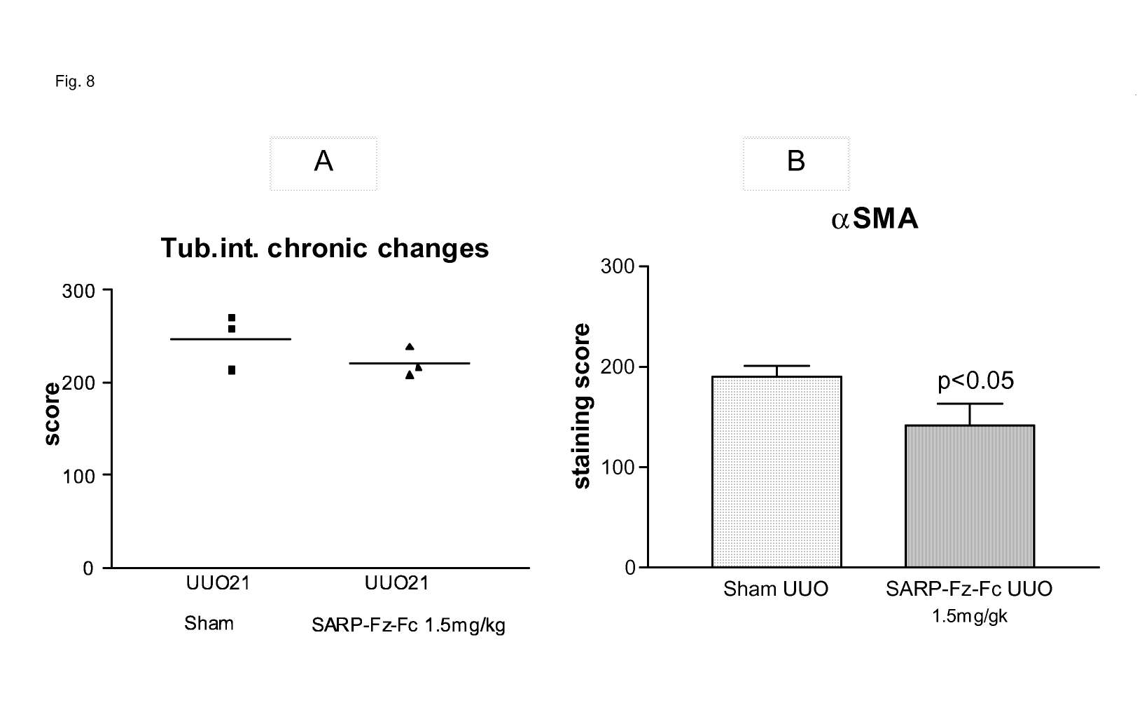

Subcutaneous injection of SARP-1(Fz)delta7-Fc at 1.5 mg/ml showed a reduction of renal fibrosis by morphometric analysis of the changes in the tubular interstitium and by histological analysis of the myofibroblast marker (α-SMA) after 21 days of treatment (p<0.05) (see FIG. 8). This data suggest a beneficial effect of SARP-1(Fz)delta7-Fc in the treatment of renal fibrosis.

### Example 8

### Effect of SARP-1(Fz)delta7-Fc on Primary Human Lung Fibroblast

In this experiment the effect of SARP-1(Fz)delta7-Fc on the activity of primary fibroblast from IPF patients was investigated.

Materials and Methods

To evaluate the effect of SARP-1(Fz)delta7-Fc, lung fibroblasts were obtained from lung explants of patients with interstitial pulmonary fibrosis (IPF) (N=3) and controls (N=3). Fibroblasts were cultured in 96 well cell culture plates in DMEM medium with high glucose (25 mM) and 10% Fetal Clone II (Hyclone) until they reached approximately 50% confluence. Fetal Clone II is an alternative to fetal bovine serum (FBS). Then the cells were cultured for 48 hours with Fetal Clone II at a concentration of 1%. Finally SARP-1(Fz)delta7-Fc at 0.1, 1 and 100 μg/ml and controls were added. Bromodeoxyuridine (5-bromo-2-deoxyuridine, BrdU) was added 24 hours later at a final concentration of 100 μM. After 24 hours of incubation, cells were fixed and lysed. Cell proliferation was evaluated with the cell proliferation ELISA BrdU colorimetric assay (Roche Diagnostics, Meylan, France) ELISA. The experiments were performed in the basal condition (without PDGF) and in the presence of PDGF (10 ng/ml), All experimental conditions were tested in triplicates and the mean value was calculated. For each experimental condition, the incorporation of BrdU was expressed as a percentage of the incorporation observed in the control condition in the same culture.

Results

In the basal condition culture with 10% Fetal Clone II, where no further stimulation was added to the cell culture medium since pathological fibroblast behave already differently than normal ones, BrdU incorporation was increased. As observed in control fibroblasts, the positive control (JNK inhibitor) reduced by 50% the incorporation of BrdU. SARP-1(Fz)delta7-Fc reduced BrdU incorporation at the highest dose tested.

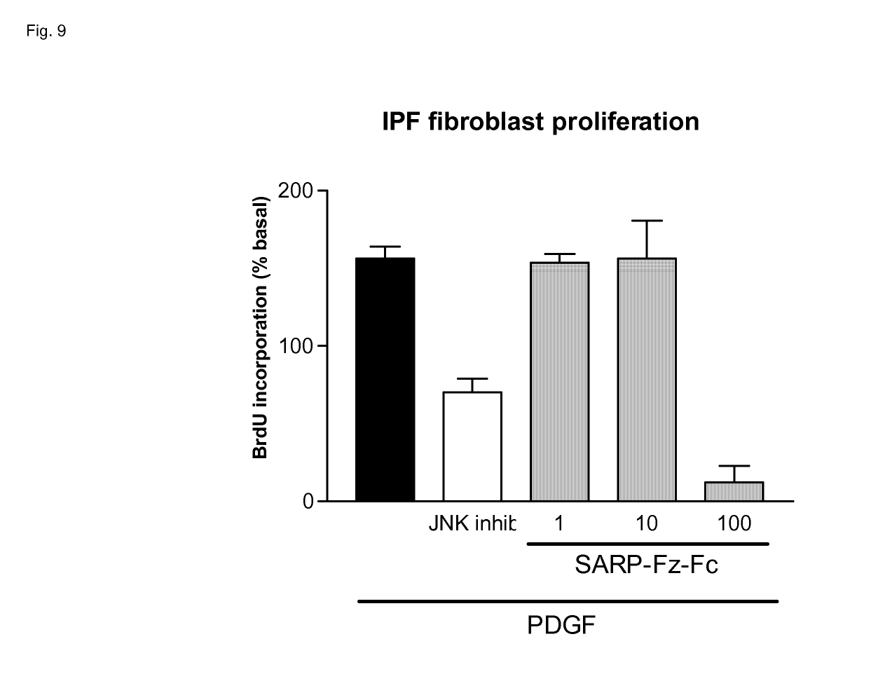

PDGF (10 ng/ml) increased BrdU incorporation by 50%; this increase was strongly inhibited by the positive control (JNK inhibitor). SARP-1(Fz)delta7-Fc had no effect at 1 and 10 μg/ml concentration but profoundly inhibited BrdU incorporation at the 100 μg/ml concentration (see FIG. 9).

### REFERENCES

Banyai, L. and Patthy, L. (1999). The NTR module: domains of netrins, secreted frizzled related proteins, and type I procollagen C-proteinase enhancer protein are homologous with tissue inhibitors of metalloproteases. Protein Sci 8:1636-1642.
Batra, S., Shi, Y., Kuchenbecker, K. M., He, B., Reguart, N., Mikami, I., You, L., Xu, Z., Lin, Y. C., Clement, G., and Jablons, D. M. (2006). Wnt inhibitory factor-1, a Wnt antagonist, is silenced by promoter hypermethylation in malignant pleural mesothelioma. Biochem Biophys Res Commun 342:1228-1232.
Bitonti, A. J. and Dumont, J. A. (2006). Pulmonary administration of therapeutic proteins using an immunoglobulin transport pathway. Adv Drug Deliv Rev 58:1106-1118.
Bitonti, A. J., Dumont, J. A., Low, S. C., Peters, R. T., Kropp, K. E., Palombella, V. J., Stattel, J. M., Lu, Y., Tan, C. A., Song, J. J., Garcia, A. M., Simister, N. E., Spiekermann, G. M., Lencer, W. I., and Blumberg, R. S. (2004). Pulmonary delivery of an erythropoietin Fc fusion protein in non-human primates through an immunoglobulin transport pathway. Proc Natl Acad Sci USA 101:9763-9768.
Caligiuri, A., De Franco, R. M., Romanelli, R. G., Gentilini, A., Meucci, M., Failli, P., Mazzetti, L., Rombouts, K., Geerts, A., Vanasia, M., Gentilini, P., Marra, F., and Pinzani, M. (2003). Antifibrogenic effects of canrenone, an antialdosteronic drug, on human hepatic stellate cells. Gastroenterology 124:504-520.
Cheng, Y. Y., Yu, J., Wong, Y. P., Man, E. P., To, K. F., Jin, V. X., Li, J., Tao, Q., Sung, J. J., Chan, F. K., and Leung, W. K. (2007). Frequent epigenetic inactivation of secreted frizzled-related protein 2 (SFRP2) by promoter methylation in human gastric cancer. Br J Cancer 97:895-901.
Chilosi, M., Poletti, V., Zamo, A., Lestani, M., Montagna, L., Piccoli, P., Pedron, S., Bertaso, M., Scarpa, A., Murer, B., Cancellieri, A., Maestro, R., Semenzato, G., and Doglioni, C. (2003). Aberrant Wnt/beta-catenin pathway activation in idiopathic pulmonary fibrosis. Am J Pathol 162:1495-1502.
Corpet, F. (1988). Multiple sequence alignment with hierarchical clustering. Dall'Acqua, W. F., Woods, R. M., Ward, E. S., Palaszynski, S. R., Patel, N. K., Brewah, Y. A., Wu, H., Kiener, P. A., and Langermann, S. (2002). Increasing the affinity of a human IgG1 for the neonatal Fc receptor: biological consequences. J Immunol 169:5171-5180.
Dann, C. E., Hsieh, J. C., Rattner, A., Sharma, D., Nathans, J., and Leahy, D. J. (2001). Insights into Wnt binding and signalling from the structures of two Frizzled cysteine-rich domains. Nature 412:86-90.
Datta-Mannan, A., Witcher, D. R., Tang, Y., Watkins, J., Jiang, W., and Wroblewski, V. J. (2007a). Humanized IgG1 variants with differential binding properties to the neonatal Fc receptor: relationship to pharmacokinetics in mice and primates. Drug Metab Dispos 35:86-94.
Datta-Mannan, A., Witcher, D. R., Tang, Y., Watkins, J., and Wroblewski, V. J. (2007b). Monoclonal antibody clearance. Impact of modulating the interaction of IgG with the neonatal Fc receptor. J Biol Chem 282:1709-1717.
Dumont, J. A., Bitonti, A. J., Clark, D., Evans, S., Pickford, M., and Newman, S. P. (2005). Delivery of an erythropoietin-Fc fusion protein by inhalation in humans through an immunoglobulin transport pathway. J Aerosol Med 18:294-303.
Dumont, J. A., Low, S. C., Peters, R. T., and Bitonti, A. J. (2006). Monomeric Fc fusions: impact on pharmacokinetic and biological activity of protein therapeutics. BioDrugs 20:151-160.
Edelman, G. M., Cunningham, B. A., Gall, W. E., Gottlieb, P. D., Rutishauser, U., and Waxdal, M. J. (1969). The covalent structure of an entire gammaG immunoglobulin molecule. Proc Natl Acad Sci USA 63:78-85.
Fischer, R., Twyman, R. M., and Schillberg, S. (2003). Production of antibodies in plants and their use for global health. Vaccine 21:820-825.
Ghetie, V., Popov, S., Borvak, J., Radu, C., Matesoi, D., Medesan, C., Ober, R. J., and Ward, E. S. (1997). Increasing the serum persistence of an IgG fragment by random mutagenesis. Nat Biotechnol 15:637-640.
Goldstein, D. A. and Thomas, J. A. (2004). Biopharmaceuticals derived from genetically modified plants. QJM 97:705-716.
He, B., Reguart, N., You, L., Mazieres, J., Xu, Z., Lee, A. Y., Mikami, I., McCormick, F., and Jablons, D. M. (2005). Blockade of Wnt-1 signaling induces apoptosis in human colorectal cancer cells containing downstream mutations. Oncogene 24:3054-3058.
He, B., You, L., Uematsu, K., Xu, Z., Lee, A. Y., Matsangou, M., McCormick, F., and Jablons, D. M. (2004). A monoclonal antibody against Wnt-1 induces apoptosis in human cancer cells. Neoplasia 6:7-14.
Hellwig, S., Drossard, J., Twyman, R. M., and Fischer, R. (2004). Plant cell cultures for the production of recombinant proteins. Nat Biotechnol 22:1415-1422.
Hinton, P. R., Johlfs, M. G., Xiong, J. M., Hanestad, K., Ong, K. C., Bullock, C., Keller, S., Tang, M. T., Tso, J. Y., Vasquez, M., and Tsurushita, N. (2004). Engineered human IgG antibodies with longer serum half-lives in primates. J Biol Chem 279:6213-6216.
Hinton, P. R., Xiong, J. M., Johlfs, M. G., Tang, M. T., Keller, S., and Tsurushita, N. (2006). An engineered human IgG1 antibody with longer serum half-life. J Immunol 176:346-356.
Huang, Z., Li, L., and Wang, J. (2007a). Hypermethylation of SFRP2 as a Potential Marker for Stool-Based Detection of Colorectal Cancer and Precancerous Lesions. Dig Dis Sci.
Huang, Z. H., Li, L. H., Yang, F., and Wang, J. F. (2007b). Detection of aberrant methylation in fecal DNA as a molecular screening tool for colorectal cancer and precancerous lesions. World J Gastroenterol 13:950-954.
Jones, S. E. and Jomary, C. (2002). Secreted Frizzled-related proteins: searching for relationships and patterns. Bioessays 24:811-820.
Jones, S. E., Jomary, C., Grist, J., Stewart, H. J., and Neal, M. J. (2000). Modulated expression of secreted frizzled-related proteins in human retinal degeneration. Neuroreport 11:3963-3967.
Kabat, E. A. (1988). Antibody complementarity and antibody structure. J Immunol 141:S25-S36.
Kamei, D. T., Lao, B. J., Ricci, M. S., Deshpande, R., Xu, H., Tidor, B., and Lauffenburger, D. A. (2005). Quantitative methods for developing Fc mutants with extended half-lives. Biotechnol Bioeng 92:748-760.
Kawano, Y. and Kypta, R. (2003). Secreted antagonists of the Wnt signalling pathway. J Cell Sci 116:2627-2634.
Kim, J. K., Firan, M., Radu, C. G., Kim, C. H., Ghetie, V., and Ward, E. S. (1999). Mapping the site on human IgG for binding of the MHC class I-related receptor, FcRn. Eur J Immunol 29:2819-2825.
Lee, A. Y., He, B., You, L., Dadfarmay, S., Xu, Z., Mazieres, J., Mikami, I., McCormick, F., and Jablons, D. M. (2004a). Expression of the secreted frizzled-related protein gene family is downregulated in human mesothelioma. Oncogene 23:6672-6676.
Lee, J. L., Chang, C. J., Wu, S. Y., Sargan, D. R., and Lin, C. T. (2004b). Secreted frizzled-related protein 2 (SFRP2) is highly expressed in canine mammary gland tumors but not in normal mammary glands. Breast Cancer Res Treat 84:139-149.
Lin, Y. C., You, L., Xu, Z., He, B., Yang, C. T., Chen, J. K., Mikami, I., Clement, G., Shi, Y., Kuchenbecker, K., Okamoto, J., Kashani-Sabet, M., and Jablons, D. M. (2007). Wnt inhibitory factor-1 gene transfer inhibits melanoma cell growth. Hum Gene Ther 18:379-386.
Low, S. C., Nunes, S. L., Bitonti, A. J., and Dumont, J. A. (2005). Oral and pulmonary delivery of FSH-Fc fusion proteins via neonatal Fc receptor-mediated transcytosis. Hum Reprod 20:1805-1813.
Melkonyan, H. S., Chang, W. C., Shapiro, J. P., Mahadevappa, M., Fitzpatrick, P. A., Kiefer, M. C., Tomei, L. D., and Umansky, S. R. (1997). SARPs: a family of secreted apoptosis-related proteins. Proc Natl Acad Sci USA 94:13636-13641.
Mikami, I., You, L., He, B., Xu, Z., Batra, S., Lee, A. Y., Mazieres, J., Reguart, N., Uematsu, K., Koizumi, K., and Jablons, D. M. (2005). Efficacy of Wnt-1 monoclonal antibody in sarcoma cells. BMC Cancer 5:53.
Mirotsou, M., Zhang, Z., Deb, A., Zhang, L., Gnecchi, M., Noiseux, N., Mu, H., Pachori, A., and Dzau, V. (2007). Secreted frizzled related protein 2 (Sfrp2) is the key Akt-mesenchymal stem cell-released paracrine factor mediating myocardial survival and repair. Proc Natl Acad Sci USA 104:1643-1648.
Muller, H. M., Oberwalder, M., Fiegl, H., Morandell, M., Goebel, G., Zitt, M., Muhlthaler, M., Ofner, D., Margreiter, R., and Widschwendter, M. (2004). Methylation changes in faecal DNA: a marker for colorectal cancer screening? Lancet 363:1283-1285.
Nikolov, Z. L. and Woodard, S. L. (2004). Downstream processing of recombinant proteins from transgenic feedstock. Curr Opin Biotechnol 15:479-486.
Nojima, M., Suzuki, H., Toyota, M., Watanabe, Y., Maruyama, R., Sasaki, S., Sasaki, Y., Mita, H., Nishikawa, N., Yamaguchi, K., Hirata, K., Itoh, F., Tokino, T., Mori, M., Imai, K., and Shinomura, Y. (2007). Frequent epigenetic inactivation of SFRP genes and constitutive activation of Wnt signaling in gastric cancer. Oncogene.
Pearson, W. R. (1990a). Rapid and sensitive sequence comparison with FASTP and FASTA. Methods Enzymol 183:63-98.
Pearson, W. R. (1990b). Rapid and sensitive sequence comparison with FASTP and FASTA. Methods Enzymol 183:63-98.
Rattner, A., Hsieh, J. C., Smallwood, P. M., Gilbert, D. J., Copeland, N. G., Jenkins, N. A., and Nathans, J. (1997). A family of secreted proteins contains homology to the cysteine-rich ligand-binding domain of frizzled receptors. Proc Natl Acad Sci USA 94:2859-2863.
Shields, R. L., Namenuk, A. K., Hong, K., Meng, Y. G., Rae, J., Briggs, J., Xie, D., Lai, J., Stadlen, A., Li, B., Fox, J. A., and Presta, L. G. (2001). High resolution mapping of the binding site on human IgG1 for Fc gamma RI, Fc gamma RII, Fc gamma RIII, and FcRn and design of IgG1 variants with improved binding to the Fc gamma R. J Biol Chem 276:6591-6604.
Shih, Y. L., Hsieh, C. B., Lai, H. C., Yan, M. D., Hsieh, T. Y., Chao, Y. C., and Lin, Y. W. (2007). SFRP1 suppressed hepatoma cells growth through Wnt canonical signaling pathway. Int J Cancer 121:1028-1035.
Surendran, K., Schiavi, S., and Hruska, K. A. (2005). Wnt-dependent beta-catenin signaling is activated after unilateral ureteral obstruction, and recombinant secreted frizzled-related protein 4 alters the progression of renal fibrosis. J Am Soc Nephrol 16:2373-2384.
Suzuki, H., Watkins, D. N., Jair, K. W., Schuebel, K. E., Markowitz, S. D., Chen, W. D., Pretlow, T. P., Yang, B., Akiyama, Y., Van Engeland, M., Toyota, M., Tokino, T., Hinoda, Y., Imai, K., Herman, J. G., and Baylin, S. B. (2004). Epigenetic inactivation of SFRP genes allows constitutive WNT signaling in colorectal cancer. Nat Genet. 36:417-422.
Tatusova, T. A. and Madden, T. L. (1999). BLAST 2 Sequences, a new tool for comparing protein and nucleotide sequences. FEMS Microbiol Lett 174:247-250.
Vaughn, D. E., Milburn, C. M., Penny, D. M., Martin, W. L., Johnson, J. L., and Bjorkman, P. J. (1997). Identification of critical IgG binding epitopes on the neonatal Fc receptor. J Mol Biol 274:597-607.
Vielhauer, V., Anders, H. J., Mack, M., Cihak, J., Strutz, F., Stangassinger, M., Luckow, B., Grone, H. J., and Schlondorff, D. (2001). Obstructive nephropathy in the mouse: progressive fibrosis correlates with tubulointerstitial chemokine expression and accumulation of CC chemokine receptor 2- and 5-positive leukocytes. J Am Soc Nephrol 12:1173-1187.
Ward, E. S, and Ghetie, V. (1995). The effector functions of immunoglobulins: implications for therapy. Ther Immunol 2:77-94.
Wheeler, D. L., Barrett, T., Benson, D. A., Bryant, S. H., Canese, K., Chetvernin, V., Church, D. M., DiCuccio, M., Edgar, R., Federhen, S., Geer, L. Y., Kapustin, Y., Khovayko, O., Landsman, D., Lipman, D. J., Madden, T. L., Maglott, D. R., Ostell, J., Miller, V., Pruitt, K. D., Schuler, G. D., Sequeira, E., Sherry, S. T., Sirotkin, K., Souvorov, A., Starchenko, G., Tatusov, R. L., Tatusova, T. A., Wagner, L., and Yaschenkou, E. (2007). Database resources of the National Center for Biotechnology Information. Nucleic Acids Res 35:D5-12.

1-18. (canceled)

19. A fusion protein comprising:
a) the mature SARP-1 polypeptide of SEQ ID NO:1, 4, 6, 8 or 10 without the Netrin domain or a variant thereof, the fusion protein further comprising the Fc region of an immunoglobulin heavy chain and wherein the fusion protein lacks the N-terminal amino acids 1-10, 1-9, 1-8, 1-7, 1-6, 1-5 or 1-4 of the mature SARP-1 polypeptide; or
b) SARP-1(Fz)delta7-Fc comprising SEQ ID NO:14 or a variant or mutein thereof.

20. The fusion protein according to claim 19, said SARP-1 polypeptide comprising amino acids 35 to 153 of SEQ ID NO:6, 8 or 10, or comprising amino acids 35 to 151 of SEQ ID NO:4.

21. The fusion protein according to claim 19, said SARP-1 polypeptide comprising amino acids 32 to 153 of SEQ ID NO:6, 8 or 10, or comprising amino acids 32 to 151 of SEQ ID NO:4.

22. The fusion protein according to claim 19, wherein the mature SARP-1 polypeptide without the Netrin domain is a SARP-1 variant, said variant having at least 70% identity with the mature SARP-1 polypeptide of SEQ ID NO:1, 4, 6, 8 or 10, and at least one of the biological activities of SARP-1.

23. The fusion protein according to claim 19, wherein the Fc region is from IgG.

24. The fusion protein according to claim 23, wherein the Fx region is from IgG₁ or IgG₄.

25. The fusion protein according to claim 19, which is:
a) SARP-1(Fz)delta7-Fc as shown in SEQ ID NO:14;
b) a variant of SARP-1(Fz)delta7-Fc, which has at least 70% identity with the polypeptide of SEQ ID NO:14 and has at least one of the biological activities of SARP-1; or
c) a mutein of SARP-1(Fz)delta7-Fc of SEQ ID NO:14, wherein not more than 150 amino acids of the polypeptide of SEQ ID NO:14 are substituted with non-conserved amino acids and said mutein has at least one of the biological activities of SARP-1.

26. A polynucleotide encoding a fusion protein according to claim 19.

27. A vector comprising the polynucleotide according to claim 26.

28. The vector according to claim 27, wherein the vector further comprises the coding sequence for the mIgSP-tPA-pro signal peptide of SEQ ID NO:24.

29. A host cell comprising a polynucleotide or vector encoding a fusion protein according to claim 19.

30. A process for preparing a fusion protein comprising expressing a fusion protein in a host cell according to claim 29 and recovering said fusion protein.

31. A method of treating a cancer, fibrotic disorder or a cardiovascular disorder comprising the administration of a therapeutically effective amount of a fusion protein according to claim 19 to an individual having a cancer, fibrotic disorder or a cardiovascular disorder.

32. The method according to claim 31, wherein the cancer is a gastrointestinal cancer, colorectal cancer, bladder cancer, pancreatic cancer, endometrial cancer, ovarian cancer, melanoma, leukemia and non-Hodgkin lymphoma, breast cancer, prostate cancer, or lung cancer.

33. The method according to claim 31, wherein the fibrotic disorder is scleroderma, unwanted or excessive scarring, lung fibrosis, liver fibrosis, fibrosis of the intestine, kidney fibrosis, heart fibrosis or skin fibrosis.

34. The method according to claim 31, wherein the cardiovascular disorder is myocardial infarction, cardiomyopathy or hypertrophic cardiomyopathy.

35. A pharmaceutical composition comprising a fusion protein according to claim 19 and a pharmaceutically acceptable carrier or excipient.
